(19) United States
(12) Patent Application Publication
(10) Pub. No.: US 2022/0152128 A1
(43) Pub. Date: May 19, 2022

(54) BEE GUT MICROBIAL FORMULATION FOR USE AS A PROBIOTIC FOR IMPROVED BEE HEALTH AND PATHOGEN RESISTANCE

(71) Applicant: Board of Regents, The University of Texas System, Austin, TX (US)

(72) Inventors: Nancy A. Moran, Austin, TX (US); Sean Leonard, Austin, TX (US); Waldan Kwong, Vancouver (CA); Jenkyn Elijah Powell, Austin, TX (US)

(21) Appl. No.: 17/431,813

(22) PCT Filed: Feb. 19, 2020
(86) PCT No.: PCT/US20/18743

§ 371 Date: Aug. 18, 2021

Related U.S. Application Data
(60) Provisional application No. 62/807,384, filed on Feb. 19, 2019.

(51) Int. Cl. A61K 35/747; A61K 35/745; A61K 35/741; A61K 9/00; A61P 1/14

(52) U.S. Cl. CPC A61K 35/747; A61K 35/745; A61P 1/14; A61K 9/0053; A61K 35/741

(57) ABSTRACT

Provided herein are defined bacterial co-cultures, and methods of generating the same. Further provided are methods of using the defined bacterial co-cultures as probiotics to prevent diseases or disorders in bees and bee colonies.

### CROSS REFERENCE TO RELATED APPLICATIONS

This application claims priority to U.S. Provisional Application No. 62/807,384, filed Feb. 19, 2019 which is hereby incorporated by reference herein in its entirety.

### STATEMENT REGARDING FEDERALLY SPONSORED RESEARCH OR DEVELOPMENT

This invention was made with government support under Grant no. 1415604 awarded by the National Science Foundation and Grant no. R01 GM108477 awarded by the National Institutes of Health and HR0011-15-C-0095 awarded by Defense Advanced Research Project Agency (DARPA). The government has certain rights in the invention.

### BACKGROUND OF THE INVENTION

Honey bees (Apis mellifera) are important agricultural pollinators. Unfortunately, recent years have seen substantial bee colony losses (e.g., Colony Collapse Disorder), due to a myriad of complex causes. Some of the most significant causes are bee viral or bacterial pathogens such as American Foulbrood (AFB) disease caused by the spore forming bacterium Paenibacillus larvae and pathogenic Serratia marcescens. There is currently no cure for some bee pathogens. Some evidence supports a role of the bee gut microbiome in supporting bee growth, bee development, bee survivorship, bee immune function, and bee resistance to several pathogens. Therefore, a disrupted bee gut microbiome can lead to bee disease and to colony declines. Many factors can disrupt a microbiome, including thermal shifts, exposure to widely used pesticides, herbicides and antibiotics, nutritional stress, pathogens and parasites and other factors. Beekeepers routinely apply antibiotics and other chemicals treatments shown to disrupt native bee flora. Currently there are few methods for curing bee diseases, and beekeepers can only take steps to prevent infections or disorders from establishing in a beekeeping operation. This invention presents a method for augmenting and improving the bee gut microbiome so as to improve the health of bees and the vigor of bee colonies, especially after the treatment with antibiotics or other stresses that disrupt the native bee gut flora.

Thus, there is an unmet need for novel methods of improving bee and colony health and pathogen resistance, and restoring the bee gut microbiome. The current invention addresses this need.

### SUMMARY OF THE INVENTION

In one embodiment, the invention relates to a bacterial co-culture comprising at least two bacterial strains of Snodgrassella alvi, Gilliamella apicola, Bartonella apis, Lactobacillus spp., and Bifidobacterium spp. In one embodiment, the co-culture comprises at least two of Snodgrassella alvi wkB2, Gilliamella apicola wkB1, Gilliamella apicola wkB7, Bartonella apis PEB0150, Lactobacillus “Firm-5” wkB10, Lactobacillus “Firm-5” wkB8 and Bifidobacterium asteroides LCep5. In one embodiment, the co-culture comprises Snodgrassella alvi wkB2 and Lactobacillus “Firm-5” wkB10, and Lactobacillus “Firm-5” wkB8.

In one embodiment, the invention relates to composition comprising an effective amount of at least two bacterial strains selected from the group consisting of Snodgrassella alvi, Gilliamella apicola, Bartonella apis, Lactobacillus spp., and Bifidobacterium spp., and a carrier. In one embodiment, the carrier is an insect comestible carrier. In one embodiment, the composition comprises at least 10³ viable bacteria cells per gram. In one embodiment, the composition comprises at least 10⁶ viable bacteria cells per gram. In one embodiment, at least one bacterial strain is in a sporulated form. In one embodiment, at least one bacterial strain is provided in a lyophilized form.

In one embodiment, the composition further comprising an antibiotic.

In one embodiment, the invention relates to an ingestible composition or supplement for bees comprising an effective amount of two bacterial strains selected from the group consisting of Snodgrassella alvi, Gilliamella apicola, Bartonella apis, Lactobacillus spp., and Bifidobacterium spp. and an insect comestible carrier. In one embodiment, the carrier is suitable for bee consumption. In one embodiment, the composition comprises at least two of Snodgrassella alvi wkB2, Gilliamella apicola wkB1, Gilliamella apicola wkB7, Bartonella apis PEB0150, Lactobacillus “Firm-5” wkB10, Lactobacillus “Firm-5” wkB8 and Bifidobacterium asteroides LCep5. In one embodiment, the composition comprises Snodgrassella alvi wkB2 and Lactobacillus “Firm-5” wkB10, and Lactobacillus “Firm-5” wkB8. In one embodiment, the ingestible composition is a pollen feed, a sucrose solution or a corn syrup solution.

In one embodiment, the invention relates to a method of treating or preventing a disease or disorder in a bee or bee colony, the method comprising administering to a bee or bee colony in need thereof a therapeutically effective amount of bacterial co-culture comprising at least two bacterial strains selected from Snodgrassella alvi, Gilliamella apicola, Gilliamella apis, Bartonella apis, Lactobacillus spp., and Bifidobacterium spp.

In one embodiment, said administering is effected at a concentration of said bacterial co-culture comprising between 10³ and 10¹⁰ viable cells in one dose.

In one embodiment, the disorder is colony collapse disorder. In one embodiment, the disorder is associated with a disruption of the normal gut microbiota due to exposure to a stress such as a chemical, temperature or nutritional stress or a viral, bacterial, fungal or protozoan.

In one embodiment, the invention relates to a method of promoting health of a bee or bee colony, the method comprising administering to the bee or bee colony a bacterial co-culture comprising at least two bacterial strains of Snodgrassella alvi, Gilliamella apicola, Gilliamella apis, Bartonella apis, Lactobacillus spp., and Bifidobacterium spp. In one embodiment, the co-culture comprises at least two of Snodgrassella alvi wkB2, Snodgrassella alvi App2-2, Snodgrassella alvi Pens2-2-5, Snodgrassella alvi Gris2-3-4, Snodgrassella alvi Snod2-1-5, Snodgrassella alvi wkB9, Snodgrassella alvi wkB273, Snodgrassella alvi wkB298, Snodgrassella alvi wkB29, Snodgrassella alvi wkB12, Snodgrassella alvi PEB0171, Snodgrassella alvi PEB0178, Snodgrassella alvi MS1-3, Gilliamella apicola wkB1, Gilliamella apicola wkB7, Gilliamella apicola wkB308, Gilliamella apicola wkB106, Gilliamella apicola wkB292, Gilliamella apicola App2-1, Gilliamella apicola wkB195, Gilliamella apicola wkB112, Gilliamella apicola wkB178, Gilliamella apicola wkB18, Gilliamella apicola wkB72, Gilliamella apicola wkB171, Gilliamella apicola wkB30, Gilliamella apicola wkB11, Gilliamella apicola PEB0154, Gilliamella apis PEB0162, Gilliamella apis PEB0183, Bartonella apis PEB0150, Lactobacillus “Firm-5” wkB10, Lactobacillus “Firm-5” wk138, Lactobacillus “Firm-4” 26254, Lactobacillus “Firm-4” 26255, and Bifidobacterium asteroides LCep5.

In one embodiment, the invention relates to a method of restoring a gut microbiome of a bee or bee colony following a disruptive episode, the method comprising administering to the bee or bee colony a bacterial co-culture comprising at least two bacterial strains of Snodgrassella alvi, Gilliamella apicola, Gilliamella apis, Bartonella apis, Lactobacillus spp., and Bifidobacterium spp. In one embodiment, the co-culture comprises at least two of Snodgrassella alvi wkB2, Snodgrassella alvi App2-2, Snodgrassella alvi Pens2-2-5, Snodgrassella alvi Gris2-3-4, Snodgrassella alvi Snod2-1-5, Snodgrassella alvi wkB9, Snodgrassella alvi wkB273, Snodgrassella alvi wkB298, Snodgrassella alvi wkB29, Snodgrassella alvi wkB12, Snodgrassella alvi PEB0171, Snodgrassella alvi PEB0178, Snodgrassella alvi MS1-3, Gilliamella apicola wkB1, Gilliamella apicola wkB7, Gilliamella apicola wkB308, Gilliamella apicola wkB106, Gilliamella apicola wkB292, Gilliamella apicola App2-1, Gilliamella apicola wkB195, Gilliamella apicola wkB112, Gilliamella apicola wkB178, Gilliamella apicola wkB18, Gilliamella apicola wkB72, Gilliamella apicola wkB171, Gilliamella apicola wkB30, Gilliamella apicola wkB11, Gilliamella apicola PEB0154, Gilliamella apis PEB0162, Gilliamella apis PEB0183, Bartonella apis PEB0150, Lactobacillus “Firm-5” wkB10, Lactobacillus “Firm-5” wkB8, Lactobacillus “Firm-4” 26254, Lactobacillus “Firm-4” 26255, and Bifidobacterium asteroides LCep5. In one embodiment, the disruptive episode is administration of an antibiotic treatment to the bee or bee colony.

In one embodiment, the invention relates to an article-of-manufacture comprising packaging material and a composition for treating or preventing a disease or disorder in a bee or bee colony being contained within said packaging material, said composition comprising a bacterial co-culture comprising at least two bacterial strains of Snodgrassella alvi, Gilliamella apicola, Bartonella apis, Lactobacillus spp., and Bifidobacterium spp. In one embodiment, the co-culture comprises at least two of Snodgrassella alvi wkB2, Snodgrassella alvi App2-2, Snodgrassella alvi Pens2-2-5, Snodgrassella alvi Gris2-3-4, Snodgrassella alvi Snod2-1-5, Snodgrassella alvi wkB9, Snodgrassella alvi wkB273, Snodgrassella alvi wkB298, Snodgrassella alvi wkB29, Snodgrassella alvi wkB12, Snodgrassella alvi PEB0171, Snodgrassella alvi PEB0178, Snodgrassella alvi MS1-3, Gilliamella apicola wkB1, Gilliamella apicola wkB7, Gilliamella apicola wkB308, Gilliamella apicola wkB106, Gilliamella apicola wkB292, Gilliamella apicola App2-1, Gilliamella apicola wkB195, Gilliamella apicola wkB112, Gilliamella apicola wkB178, Gilliamella apicola wkB18, Gilliamella apicola wkB72, Gilliamella apicola wkB171, Gilliamella apicola wkB30, Gilliamella apicola wkB11, Gilliamella apicola PEB0154, Gilliamella apis PEB0162, Gilliamella apis PEB0183, Bartonella apis PEB0150, Lactobacillus “Firm-5” wkB10, Lactobacillus “Firm-5” wkB8, Lactobacillus “Firm-4” 26254, Lactobacillus “Firm-4” 26255, and Bifidobacterium asteroides LCep5.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 depicts exemplary experimental results demonstrating the relative abundance of bacteria in colonized bees. Bars correspond to bacteria colonizing individual bee guts over 12 days of trial, as determined by destructive sampling and 16S rRNA gene profiling.

FIG. 2 depicts exemplary experimental results demonstrating co-culture inoculated bee gut microbiomes are more similar than separate-culture inoculated bees. Weighted unifrac distance between samples of co-culture and separate culture bees (all samples from FIG. 1). Each point represents the gut microbiome of an individual bee. Points from co-culture bees are clustered closer together (more similar), than points from separate-culture bees.

FIG. 3 depicts exemplary experimental results demonstrating that defined community recapitulates bee weight gain from normal bee gut bacteria.

FIG. 4 depicts exemplary experimental results demonstrating that defined community recapitulates changes in gene expression associated with normal bee gut bacteria.

FIG. 5 depicts exemplary experimental results demonstrating bee survival after low-dose Serratia exposure. Bees fed probiotic cocktail show improved survival 7 days after treatment.

FIG. 6 depicts exemplary experimental results demonstrating bee survival after high-dose Serratia exposure. Bees fed probiotic cocktail show significantly improved survival 7 days after treatment.

FIG. 7 depicts exemplary experimental results demonstrating a survival curve of acute oxytetracycline treated bees after exposure to S. marcescens. Condition=Tet45_DC is for bees treated with a probiotic mixture of bee gut microbiota prior to antibiotic exposure. Shading indicates 95% confidence intervals. Statistical analysis via Cox Proportional Hazards Model demonsrated a significant difference between the conditions (p<0.05).

FIG. 8 depicts exemplary experimental results demonstrating a survival curve of tylosine tartarate treated bees after exposure to S. marcescens. Condition=tyl25_DC is for bees treated with a probiotic mixture of bee gut microbiota prior to antibiotic exposure. Shading indicates 95% confidence intervals. Statistical analysis via Cox Proportional Hazards Model demonstrated a significant difference between the conditions (p<0.05).

FIG. 9 depicts exemplary bacterial challenge experimental results demonstrating that Tylosin treated hives had bees with lower survival after bacterial challenge than did bees from control hives.

FIG. 10 depicts exemplary bacterial challenge experimental results demonstrating that treatment of bees with probiotic mixture after antibiotic treatment increased survival significantly.

FIG. 11 depicts exemplary experimental results demonstrating that bees treated with probiotic mix exhibited pronounced upregulation of immunity related genes within hours of treatment

FIG. 12 depicts an exemplary survival assay demonstrating that bees colonized with wkB2 isolates demonstrated significantly higher survival rate against a pathogen (Serratia strain N10A28).

FIG. 13 depicts an exemplary survival assay demonstrating that bees colonized with Firm-5 and a defined bacterial community (DC) demonstrated significantly higher survival rate against a pathogen (Serratia strain N10A28).

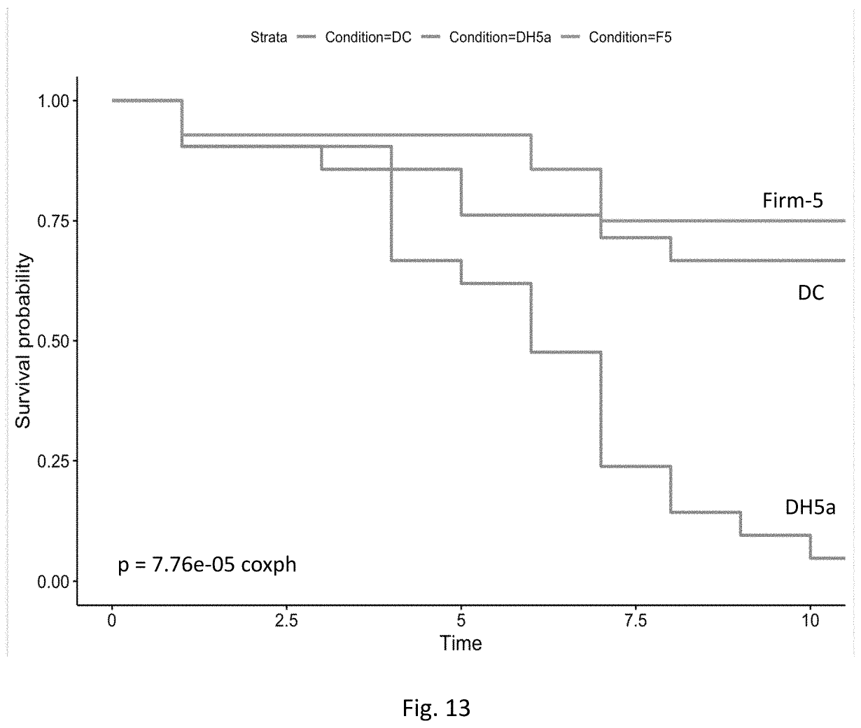

FIG. 14 depicts exemplary experimental results demonstrating that Bees colonized with specific combinations of probiotic isolates demonstrate significantly lower infection levels after infection with the pathogen (Serratia strain KZ11,“SnM”).

FIG. 15 depicts exemplary experimental results demonstrating the S. marcescens kz11 abundance in the midgut and hindgut of microbiota-free bees (MF), bees with a conventional gut microbiota (CV), and conventionalized bees treated with tetracycline (Tet) one day after oral exposure to S. marcescens.

FIG. 16 depicts exemplary experimental results demonstrating the fraction of MF and CV bees infected with S. marcescens (top) and the abundance of S. marcescens in the midgut and hindgut (bottom) one, two, three, or four days after oral exposure to S. marcescesns kz11.

FIG. 17 depicts exemplary experimental results demonstrating that “conventional” communities differ in ability to confer resistance to S. marcescens. Age-controlled, microbiota-free honey bees from hive 6 were inoculated with gut homogenate from a nurse bee from hive 1 (CV community 1) or hive 4 (CV community 2). After five data, bees were exposed to WT or ΔtssE1ΔtssE2 S. marcescens. The fraction of bees infected and the abundance of S. marcescens in the midgut and hindgut were measured 10 days after exposure.

FIG. 18 depicts exemplary experimental results demonstrating that bee gut isolates confer resistance to colonization of the gut by S. marcescens. Microbiota-free bees were inoculated with representative strains of core gut taxa: Lactobacillus Firm-5 (wkB8 and wkB10), Lactobacillus Firm-4 (26254 and 26255), Snodgrassella alvi (wkB2), Gilliamella sp. (G. apicola wkB1 and PEB0154, G. apis PEB0162 and PEB0183).

### DETAILED DESCRIPTION

The present invention is directed to compositions and methods for the biological control of the welfare of bees, and for prophylaxis and treatment of pathological disorders of bees. In one embodiment, the composition comprises a defined bacterial culture comprising at least two bacterial strains native to the bee gut. Exemplary bacterial species native to the bee gut include, but are not limited to, Snodgrassella alvi, Gilliamella apicola, Gilliamella apis, Bartonella apis, Lactobacillus spp., and Bifidobacterium spp. Therefore, in certain aspects the present invention provides defined bacterial co-cultures comprising at least two bacterial strains of Snodgrassella alvi, Gilliamella apicola, Gilliamella apis, Bartonella apis, Lactobacillus spp., and Bifidobacterium spp.

In one embodiment, the defined bacterial co-culture of the invention comprises at least two bacterial species native to the bee gut which have been combined and propagated as a single culture. Therefore, in one embodiment, the defined bacterial co-culture of the invention is generated through a process in which at least two isolated bacterial strains of Snodgrassella alvi, Gilliamella apicola, Gilliamella apis, Bartonella apis, Lactobacillus spp., and Bifidobacterium spp are combined and cultured in a single culture.

The invention also provides methods of using the defined bacterial co-culture compositions as probiotics for the prevention of diseases or disorders of bees or bee colonies, including, but not limited to colony collapse disorder, diseases or disorders associated with a viral or bacterial bee pathogen, including Deformed Wing Virus (DWV) and other viral pathogens, and also including opportunistic bacterial pathogens of adult worker bees, such as S. marcescens and other Enterobacteriaceae pathogens, and also including protozoan parasites such as Nosema species or Crithidia species. The invention may also protect against larval disease, including fungal pathogens such as chalkbrood and bacterial disease, such as American Foulbrood (AFB) disease and parasites such as Varroa mites. This invention may also improve health of bees in which the gut microbiota is perturbed due to exposure to chemicals including glyphosate or antibiotics or other chemicals, exposure to nutritional stress, exposure to toxic molecules present in hives or in pollen or nectar collected by bees, exposure to food supplements provided to hives by bee keepers, and exposure to other factors affecting the microbiota. This invention may also improve the health of bees that have not been exposed to particular stressors, by making them more robust to variability in environmental conditions.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

An “effective amount” or “therapeutically effective amount” of a compound is that amount of a compound which is sufficient to provide a beneficial effect to the subject to which the compound is administered.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.01%. Most preferred is a composition in which no amount of the specified component can be detected with standard analytical methods. “Genetically engineered bacteria” refers to bacterial cells that replicate a heterologous nucleic acid, or express a polypeptide encoded by a heterologous nucleic acid.

“Heterologous nucleic acid” is one that originates from a source foreign to the particular host cell, or, if from the same source, is modified from its original form.

As used herein “increasing host fitness” or “promoting host fitness” refers to any favorable alteration in host physiology, or any activity carried out by said host, including, but not limited to, any one or more of the following desired effects: (1) increasing a population of a host by about 10%, 20%, 30%, 40%, 50%, 60%, 70%, 80%, 90%, 95%, 99%, 100% or more; (2) increasing the reproductive rate of a host (e.g., bee) by about 10%, 20%, 30%, 40%, 50%, 60%, 70%, 80%, 90%, 95%, 99%, 100% or more; (3) increasing the mobility of a host (e.g., bee) by about 10%, 20%, 30%, 40%, 50%, 60%, 70%, 80%, 90%, 95%, 99%, 100% or more; (4) increasing the body weight of a host (e.g., bee) by about 10%, 20%, 30%, 40%, 50%, 60%, 70%, 80%, 90%, 95%, 99%, 100% or more; (5) increasing the metabolic rate or activity of a host (e.g., bee) by about 10%, 20%, 30%, 40%, 50%, 60%, 70%, 80%, 90%, 95%, 99%, 100% or more; (6) increasing pollination (e.g., number of plants pollinated in a given amount of time) by a host (e.g., bee) by about 10%, 20%, 30%, 40%, 50%, 60%, 70%, 80%, 90%, 95%, 99%, 100% or more; or (7) increasing production of host (e.g., bee) byproducts (e.g., honey from a honeybee) by about 10%, 20%, 30%, 40%, 50%, 60%, 70%, 80%, 90%, 95%, 99%, 100% or more. An increase in host fitness can be determined in comparison to a host organism to which the defined bacterial co-culture composition has not been administered.

As used herein, an “instructional material” includes a publication, a recording, a diagram, or any other medium of expression which can be used to communicate the usefulness of a compound, composition, vector, or system of the invention in the kit. Optionally, or alternately, the instructional material can describe one or more methods of modulating expression of a gene product using a compound, composition, vector, or system of the invention in the kit. The instructional material of the kit of the invention can, for example, be affixed to a container which contains the identified compound, composition, vector, or delivery system of the invention or be shipped together with a container which contains the identified compound, composition, vector, or system. Alternatively, the instructional material can be shipped separately from the container with the intention that the instructional material and the kit be used cooperatively by the recipient.

As used herein, the term “bee” is defined as any of several winged, hairy-bodied, usually stinging insects of the superfamily Apoidea in the order Hymenoptera, including both solitary and social species and characterized by sucking and chewing mouthparts for gathering nectar and pollen. Exemplary bee species include, but are not limited to species in the genera Apis, Bombus, Trigona, Osmia and the like. In one embodiment, bees include, but are not limited to bumblebees (Bombus terrestris, Bombus impatiens, or other Bombus species) and honeybees (Apis mellifera or Apis cerana).

As used herein, the term “colony” is defined as a population of dozens to typically several tens of thousands of honeybees that cooperate in nest building, food collection, and brood rearing. A colony normally has a single queen, the remainder of the bees being either “workers” (females) or “drones” (males). The social structure of the colony is maintained by the queen and workers and depends on an effective system of communication. Division of labor within the worker caste primarily depends on the age of the bee but varies with the needs of the colony. Reproduction and colony strength depend on the queen, the quantity of food stores, and the size of the worker force. Honeybees can also be subdivided into the categories of “hive bees”, usually for the first part of a worker's lifetime, during which the “hive bee” performs tasks within the hive, and “forager bee”, during the latter part of the bee's lifetime, during which the “forager” locates and collects pollen and nectar from outside the hive, and brings the nectar or pollen into the hive for consumption and storage. The term “colony” can also refer to a colony of bumble bees (Bombus species), which may also include a queen and from a few to hundreds of workers, that cooperate in nest building, rearing brood, and food collection.

As used herein, the term “plant” refers to whole plants, plant organs, plant tissues, seeds, plant cells, seeds, and progeny of the same. Plant cells include, without limitation, cells from seeds, suspension cultures, embryos, meristematic regions, callus tissue, leaves, roots, shoots, gametophytes, sporophytes, pollen, or microspores. Plant parts include differentiated or undifferentiated tissues including, but not limited to the following: roots, stems, shoots, leaves, pollen, seeds, tumor tissue, and various forms of cells and culture (e.g., single cells, protoplasts, embryos, or callus tissue). The plant tissue may be in a plant or in a plant organ, tissue, or cell culture.

As used herein, the term “susceptibility” is defined as the ability of a bee or bee colony to become infested or infected by and/or support proliferation of a pathogen, including, but not limited to, degree of infection, severity of symptoms, infectivity to other individuals (contagion), and the like. Susceptibility can be assessed, for example, by monitoring infectivity, presence of symptoms, such as, but not limited to, hunger, vitality, flight range, etc, presence of pathogenic organisms, mortality or time course of a disease in an individual bee or bee population following a challenge with the pathogen.

As used herein, the terms “bee disease” or “bee colony disease” are defined as undesirable changes in the behavior, physiology, morphology, reproductive fitness, economic value, viability, honey production, pollination capability, resistance to infection and/or infestation of a bee, a population of bees and/or a bee colony, directly or indirectly resulting from contact with a pathogen, parasite or an infected bee or other organism.

The terms “subject,” “individual,” and the like are used interchangeably herein, and refer to any animal, or cells thereof whether in vitro or in situ, amenable to the methods described herein. In certain non-limiting embodiments, subject or individual is a bee.

“Sample” or “biological sample” as used herein means a biological material isolated from a subject. The biological sample may comprise cellular and/or non-cellular material obtained from the subject. One example of a biological sample is a tissue sample.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

A “therapeutic” treatment is a treatment administered to a subject who exhibits signs or symptoms of a disease or disorder, for the purpose of diminishing or eliminating those signs or symptoms.

As used herein, “treating a disease or disorder” means reducing the severity and/or frequency with which a sign or symptom of the disease or disorder is experienced by a subject.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The honeybee has several characteristic bacterial species that together comprise over 95% of the gut bacteria in healthy adult worker bees. Bacterial species known to colonize the honeybee gut microbiota, include, but are not limited to, Snodgrassella alvi, Gilliamella apicola, Bartonella apis, species of Firmicutes, and Bifidobacteriaceae. The invention is based, in part, on the generation of a defined culture comprising two or more native gut species that can be used as a probiotic formulation to promote bee health, or bee colony health. In one embodiment, the probiotic formulation prevents pathogenesis in bees.

In one embodiment, the invention provides defined bacterial co-cultures comprising two or more native bacterial gut species. In one embodiment, the invention provides defined bacterial co-cultures comprising two or more engineered bacteria, wherein the engineered bacteria are from two or more native bacterial gut species.

In one embodiment, the invention provides methods of use of the defined bacterial co-cultures to treat or prevent a bee or bee colony disease or disorder. In one embodiment, the disease or disorder is associated with a bee or bee colony parasite or pathogen.

### Compositions

In part, the present invention is directed to compositions for the biological control of the welfare of bees, and for prophylaxis and treatment of pathological disorders of bees. In some embodiments, the compositions described herein includes one or more bacteria. Numerous bacteria are useful in the compositions and methods described herein. In some instances, the bacteria is a bacterial species endogenously found in the host. In some instances, the bacteria is a symbiotic bacterial species. Non-limiting examples of bacteria that may be used in defined bacterial co-culture compositions of the invention include, but are not limited to, bacterial species from any bacterial phyla present in bee guts, including Gammaproteobacteria, Alphaproteobacteria, Betaproteobacteria, Bacteroidetes, Firmicutes (e.g., Lactobacillus and Bacillus spp.), Clostridia, Actinomycetes, Spirochetes, Verrucomicrobia, and Actinobacteria.

In some instances, the bacteria is a bacterium that promotes microbial diversity or otherwise alters the microbiota of the host in a favorable manner. In one instance, bacteria may be provided to promote microbiome development in honey bees. For example, the bacteria may include, for example, Bartonella apis, Parasaccharibacter apium, Frischella perrara, Snodgrassella alvi, Gilliamela apicola, Gilliamela apis, Bifidobacterium spp, or Lactobacillus spp.

The compositions discussed herein can be used to alter the level, activity, or metabolism of target microorganisms as indicated in the sections for increasing the fitness of insects, such as, honeybees.

In one embodiment, the composition comprises a defined bacterial culture comprising at least two bacterial strains native to the bee gut. In one embodiment, the at least two bacterial strains are from the same species of bacterium. In one embodiment, the composition comprises at least two bacterial strains, wherein each strain is from a different species of bacterium. In one embodiment, one or more bacterial strain included in the defined bacterial co-culture is a strain with properties suitable to confer tolerance to particular exposures or provide advantage in particular situations. Potential sources of gut microbiome disruption include antibiotic treatment, and pesticide exposure or herbicide exposure (e.g., glyphosate). Therefore, in various embodiments, one or more bacterial strain included in the defined bacterial co-culture is a strain with properties suitable to confer tolerance to antibiotic treatment, and pesticide exposure or herbicide exposure. As a non-limiting example, S. alvi wkB2 is resistant to tetracycline and tolerant of glyphosate, therefore, in one embodiment, the defined bacterial co-culture comprises S. alvi wkB2 to confer tolerance to glyphosate and resistance to tetracycline exposure.

In one embodiment, the composition comprises a defined bacterial culture comprising at least two bacterial strains, wherein each strain is from S. alvi, G. apicola, G. apis, Bartonella apis, Lactobacillus spp., or Bifidobacterium spp. In one embodiment, the composition comprises a defined bacterial culture comprising at least 2, 3, 4, 5, 6, 7, 8, 9, 10 or more than 10 bacterial strains, wherein each strain is from S. alvi, G. apicola, G. apis, Bartonella apis, Lactobacillus spp., or Bifidobacterium spp. In one exemplary embodiment, the composition comprises 2 bacterial strains from Lactobacillus spp. In one exemplary embodiment, the composition comprises 3 bacterial strains, wherein 2 bacterial strains are from Lactobacillus spp, and 1 bacterial strain is from S. alvi. In one exemplary embodiment, the composition comprises 4 bacterial strains, wherein 2 bacterial strains are from G. apicola and 2 bacterial strains are from G. apis.

In one embodiment, the composition comprises a defined bacterial culture comprising at least two bacterial species of S. alvi, G. apicola, G. apis, Bartonella apis, Lactobacillus spp., and Bifidobacterium spp. In one embodiment, the composition comprises a defined bacterial culture comprising at least three bacterial species selected from S. alvi, G. apicola, G. apis, Bartonella apis, Lactobacillus spp., and Bifidobacterium spp. In one embodiment, the composition comprises a defined bacterial culture comprising at least four bacterial species selected from S. alvi, G. apicola, G. apis, Bartonella apis, Lactobacillus spp., and Bifidobacterium spp. In one embodiment, the composition comprises a defined bacterial culture comprising at least five bacterial species selected from S. alvi, G. apicola, G. apis, Bartonella apis, Lactobacillus spp., and Bifidobacterium spp. In one embodiment, the composition comprises a defined bacterial culture comprising at least six bacterial species selected from S. alvi, G. apicola, G. apis, Bartonella apis, Lactobacillus spp., and Bifidobacterium spp. In one embodiment, the composition comprises a defined bacterial culture comprising more than five bacterial species selected from S. alvi, G. apicola, G. apis, Bartonella apis, Lactobacillus spp., and Bifidobacterium spp.

Exemplary strains of bacteria that can be included in a defined culture of the invention include, but are not limited to, Snodgrassella alvi wkB2, Snodgrassella alvi App2-2, Snodgrassella alvi Pens2-2-5, Snodgrassella alvi Gris2-3-4, Snodgrassella alvi Snod2-1-5, Snodgrassella alvi wkB9, Snodgrassella alvi wkB273, Snodgrassella alvi wkB298, Snodgrassella alvi wkB29, Snodgrassella alvi wkB12, Snodgrassella alvi PEB0171, Snodgrassella alvi PEB0178, Snodgrassella alvi MS1-3, Gilliamella apicola wkB1, Gilliamella apicola wkB7, Gilliamella apicola wkB308, Gilliamella apicola wkB106, Gilliamella apicola wkB292, Gilliamella apicola App2-1, Gilliamella apicola wkB195, Gilliamella apicola wkB112, Gilliamella apicola wkB178, Gilliamella apicola wkB18, Gilliamella apicola wkB72, Gilliamella apicola wkB171, Gilliamella apicola wkB30, Gilliamella apicola wkB11, Gilliamella apicola PEB0154, Gilliamella apis PEB0162, Gilliamella apis PEB0183, Bartonella apis PEB0150, Lactobacillus “Firm-5” wkB10, Lactobacillus “Firm-5” wkB8, Lactobacillus “Firm-4” 26254, Lactobacillus “Firm-4” 26255, and Bifidobacterium asteroides LCep5.

In one embodiment, the defined bacterial co-culture comprises Snodgrassella alvi wkB2, Gilliamella apicola wkB1, Gilliamella apicola wkB7, Bartonella apis PEB0150, Lactobacillus “Firm-5” wkB10, and Lactobacillus “Firm-5” wkB8. In one embodiment, the defined bacterial co-culture comprises Snodgrassella alvi wkB2, Gilliamella apicola wkB1, Gilliamella apicola wkB7, Lactobacillus “Firm-5” wkB10, and Lactobacillus “Firm-5” wkB8. In one embodiment, the defined bacterial co-culture comprises Lactobacillus “Firm-5” wkB10, and Lactobacillus “Firm-5” wkB8. In one embodiment, the defined bacterial co-culture comprises Snodgrassella alvi wkB2, Lactobacillus “Firm-5” wkB10, and Lactobacillus “Firm-5” wkB8. In one embodiment, the defined bacterial co-culture comprises Gilliamella apicola wkB1, Gilliamella apicola PEB0154, Gilliamella apis PEB0162, and Gilliamella apis PEB0183. In one embodiment, the defined bacterial co-culture comprises Snodgrassella alvi wkB2, Lactobacillus “Firm-5” wkB10, and Lactobacillus “Firm-5” wkB8, Gilliamella apicola wkB1, Gilliamella apis PEB0162 and Bifidobacterium asteroides LCep5.

In various embodiments, the bacterial co-culture of the present invention may include other strains of probiotic bacteria, yeast or mold. Examples of probiotic bacterial strains include but are not limited to the Lactobacillus genus including, but not limited to, Lactobacillus kunkeei, Lactobacillus apinorum, Lactobacillus mellifer, Lactobacillus mellis, Lactobacillus melliventris, Lactobacillus kimbladii, Lactobacillus kullabergensis. Other examples of probiotic bacterial strains may include other strains or species of the genus Snodgrassella, other strains or species of the genus Gilliamella, or of Parasaccharibacter apium or other species of Parasaccharibacter, and strains of Acetobacteriaceae referred to as “Alpha 2.2” and “Alpha 2.1”. Other probiotic bacterial strains include strains of Frischella perrara, Serratia marcescens, and Schmidhempelia species. Other potential strains include Lactobacillus plantarum, Lactobacillus salivarius, Lactobacillus delbrukil, Lactobacillus rhamnosus, Lactobacillus bulgaricus, Lactobacillus gaserli, Lactobacillus jensenii and Lactobacillus sporogenes; the Enterococccus genus, including Enterococcus faecium and Enterococcus thermophilus; the Bifidobacterium genus, including Bifidobacterium longum, Bifidobacterium infantis, and Bifidobacterium bifidum; Bacillus genus, including Bacillus coagulans, Bacillus thermophilus, Bacillus laterosporus, Bacillus subtilis, Bacillus megaterium, Bacillus licheniformis, Bacillus mycoides, Bacillus pumilus, Bacillus lentus, Bacillus cereus and Bacillus circulans; Pseudomonas genus, including Pseudomonas aeruginosa, Pseudomonas putida, Pseudomonas cepacia, Pseudomonas fluorescens, and Pseudomonas 679-2; Sporolactobacillus genus; Micromonospora genus; Micrococcus genus; Rhodococcus genus and Escherichia coli.

In one embodiment, one or more of the bacterial species in the composition are spore forming species. Therefore, in one embodiment, the composition may comprise one or more bacterial species in sporulated form.

In one embodiment, the defined culture is useful as a probiotic for promoting microbiome development in bees, including, but not limited to bumblebees (Bombus terrestris and other Bombus species), honeybees (Apis mellifera) (including foragers and hive bees) and Apis cerana.

The defined bacterial co-culture compositions and products described above may include live bacteria, lyophilized bacteria or killed bacteria. Furthermore, compositions and products may include metabolites and/or bacteriocins produced. Products containing lyophilized bacterial strains, can be activated by the addition of water or water containing nutrients.

In one embodiment, the composition of the invention comprises viable bacterial cells from at least two bacterial strains. In one embodiment, the composition comprises 10³ to 10¹³ viable cells/gram. In various embodiments, the composition comprises at least about 10³, at least about 10⁴, at least about 10⁵, at least about 10⁶, or more than 10⁶ viable cells/gram. In one embodiment, the composition comprises 10³ to 10¹³ viable cells/mL. In various embodiments, the composition comprises at least about 10³, at least about 10⁴, at least about 10⁵, at least about 10⁶, or more than 10⁶ viable cells/mL.

It will be appreciated that besides viable cells, non-viable cells such as killed cultures or compositions containing beneficial factors expressed by the probiotic bacteria of the present invention can also be administered. This could include thermally killed cells or bacterial cells killed by exposure to altered pH or subjection to pressure. It will be appreciated that compositions including non-viable bacterial products are simpler to generate and store.

In one embodiment, the composition comprises at least two bacterial strains wherein the first bacterial strain represents at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40% at least 45%, at least 50%, at least 60%, at least 65%, at least 70%, at least 75%, at least 80%, at least 85%, at least 90%, at least 95% or more than 95% of the total bacteria present in the composition, and the second bacterial strain represents at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40% at least 45%, at least 50%, at least 60%, at least 65%, at least 70%, at least 75%, at least 80%, at least 85%, at least 90%, at least 95% or more than 95% of the total bacteria present in the composition. For example, in one embodiment, the ratio of two bacterial strains may be 1:99, 99:1, or any ratio therebetween.

In one embodiment, the composition comprises at least three bacterial strains wherein the first bacterial strain represents at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40% at least 45%, at least 50%, at least 60%, at least 65%, at least 70%, at least 75%, at least 80%, at least 85%, at least 90%, at least 95% or more than 95% of the total bacteria present in the composition, wherein the second bacterial strain represents at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40% at least 45%, at least 50%, at least 60%, at least 65%, at least 70%, at least 75%, at least 80%, at least 85%, at least 90%, at least 95% or more than 95% of the total bacteria present in the composition, and wherein the third bacterial strain represents at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40% at least 45%, at least 50%, at least 60%, at least 65%, at least 70%, at least 75%, at least 80%, at least 85%, at least 90%, at least 95% or more than 95% of the total bacteria present in the composition. For example, in one embodiment, the ratio of three bacterial strains may be 1:1:98, 1:98:1, 98:1:1, or any ratio therebetween.

In one embodiment, the composition comprises at least four bacterial strains wherein the first bacterial strain represents at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40% at least 45%, at least 50%, at least 60%, at least 65%, at least 70%, at least 75%, at least 80%, at least 85%, at least 90%, at least 95% or more than 95% of the total bacteria present in the composition, wherein the second bacterial strain represents at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40% at least 45%, at least 50%, at least 60%, at least 65%, at least 70%, at least 75%, at least 80%, at least 85%, at least 90%, at least 95% or more than 95% of the total bacteria present in the composition, wherein the third bacterial strain represents at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40% at least 45%, at least 50%, at least 60%, at least 65%, at least 70%, at least 75%, at least 80%, at least 85%, at least 90%, at least 95% or more than 95% of the total bacteria present in the composition, and wherein the fourth bacterial strain represents at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40% at least 45%, at least 50%, at least 60%, at least 65%, at least 70%, at least 75%, at least 80%, at least 85%, at least 90%, at least 95% or more than 95% of the total bacteria present in the composition. For example, in one embodiment, the ratio of four bacterial strains may be 1:1:1:97, 1:1:97:1, 1:97:1:1, 97:1:1:1 or any ratio therebetween. Similarly, for five bacterial strains the ratio may be 1:1:1:1:96, 1:1:1:96:1, 1:1:96:1:1, 1:96:1:1:1, 96:1:1:1:1 or any ratio therebetween, and for six bacterial strains the ratio may be 1:1:1:1:1:95, 1:1:1:1:95:1, 1:1:1:95:1:1, 1:1:95:1:1:1, 1:95:1:1:1:1, 95:1:1:1:1:1 or any ratio therebetween.

In one embodiment, the defined bacterial co-culture comprises one or more bacterial strains that has been modified to express a heterologous nucleic acid sequence including, but not limited to, a heterologous DNA or RNA sequence. In one embodiment, the heterologous DNA or RNA molecule is useful for protecting a bee or bee colony from a disease or disorder (e.g., a siRNA targeting a gene of a bee or bee colony pathogen.) Methods of modifying bacterial species for expression of heterologous nucleic acid sequences are known in the art. Examples of such heterologous sequences include DNA sequences encoding double stranded RNA or siRNA that would target genes of bee pathogens, including viral pathogens such as Deformed Wing Virus and Israeli Acute Paralysis Virus, protozoan parasites such as species of Nosema or Crithidia, and arthropod pathogens such as Varroa mites and Small Hive Beetles.

In some embodiments, the composition further includes an agent that alters a level, activity, or metabolism of one or more microorganisms resident in an insect host, the alteration resulting in an increase in the insect host's fitness. In some embodiments, the agent is a polypeptide, a small molecule, an antibiotic, a bacterium, or any combination thereof.

Antibiotics

The compositions of the present invention may include a therapeutically-effective amount of an antibiotic. Measures are taken to include an antibiotic or a concentration thereof, which does not affect the bacterial strains of the present invention. For example, the bacterial strains of the present invention may be combined with a therapeutic dose of an antibiotic such as lincomycin, oxytetracycline or tylosine tartarate. However, other antibiotics or secondary components can also be used according to this aspect of the present invention. Exemplary antibiotics and secondary components that can be included in a composition of the invention include, but are not limited to lincomycin, oxytetracycline, tylosine tartarate, fumagillin, amitraz, oxalic acid, thymol, or natural plant-derived compounds or mixtures of compounds.

### Formulations

The compositions described herein may be formulated either in pure form (e.g., the composition contains only the defined bacterial co-culture) or together with one or more additional agents (such as excipient, adjuvant, etc.) to facilitate application or delivery of the compositions.

In one embodiment, the composition will comprise at least two bacterial strains selected from S. alvi, G. apicola, G. apis, Bartonella apis, Lactobacillus spp., and Bifidobacterium spp, and an acceptable carrier. Such a composition can be in the form of for example, a liquid suspension, a paste, a syrup, or a gel. An acceptable carrier should be non-toxic to the bacterial species included in the defined bacterial co-culture and to the bees to which it is to be administered, and can also include an ingredient that promotes viability of the microorganisms during storage. The carrier can be, for example, a liquid carrier or gel-based carrier, which are well known in the art. Such carriers include, but are not limited to, water, physiological electrolyte solutions, and glycols such as methanol, ethanol, propanol, butanol, ethylene glycol, and propylene glycol. In one embodiment, the carrier is an insect comestible carrier as a liquid, a solid, an aerosol, a paste, a gel, or a gas. In one embodiment, the carrier is suitable for bee consumption.

The composition can further comprise one or more carbon sources as a nutrient source for the bees, such as fructose, glucose, sucrose, maltose, galactose, sorbitol, xylan, pectin, and lignin. In particular examples, the carbon source is at least one of sucrose, fructose, and glucose.

In some embodiments, the composition is a bee-ingestible composition. In certain aspects, the bacteria are present as a live suspension or a lyophilized powder. The composition may be in solid form or liquid form, such as a sucrose solution or a corn syrup solution. In some aspects, the composition comprises protein and/or pollen.

In some compositions, the composition may further include a host bait, a sticky agent, or a combination thereof. In some embodiments, the host bait is a comestible agent and/or a chemoattractant.

In some embodiments, the composition may be formulated for delivery to the gut of the host. In some embodiments, the composition may be formulated for use in a host feeding station.

Examples of suitable excipients and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, talc, magnesium stearate, and mineral oil.

In some instances, the composition includes a delivery vehicle or carrier. In some instances, the delivery vehicle includes an excipient. Exemplary excipients include, but are not limited to, solid or liquid carrier materials, solvents, stabilizers, slow-release excipients, colorings, and surface-active substances (surfactants). In some instances, the delivery vehicle is a stabilizing vehicle. In some instances, the stabilizing vehicle includes a stabilizing excipient. Exemplary stabilizing excipients include, but are not limited to, epoxidized vegetable oils, antifoaming agents, e.g. silicone oil, preservatives, viscosity regulators, binding agents and tackifiers. In some instances, the stabilizing vehicle is a buffer suitable for the defined bacterial co-culture composition. In some instances, the composition is microencapsulated in a polymer bead delivery vehicle. In some instances, the stabilizing vehicle protects the defined bacterial co-culture composition against UV and/or acidic conditions. In some instances, the delivery vehicle contains a pH buffer. In some instances, the composition is formulated to have a pH in the range of about 4.5 to about 9.0, including for example pH ranges of about any one of 5.0 to about 8.0, about 6.5 to about 7.5, or about 6.5 to about 7.0.

Depending on the intended objectives and prevailing circumstances, the composition may be formulated into emulsifiable concentrates, suspension concentrates, directly sprayable or dilutable solutions, coatable pastes, diluted emulsions, spray powders, soluble powders, dispersible powders, wettable powders, dusts, granules, encapsulations in polymeric substances, microcapsules, foams, aerosols, carbon dioxide gas preparations, tablets, resin preparations, paper preparations, nonwoven fabric preparations, or knitted or woven fabric preparations. In some instances, the composition is a liquid. In some instances, the composition is a solid. In some instances, the composition is an aerosol, such as in a pressurized aerosol can. In some instances, the composition is present in the waste (such as feces) of the pest. In some instances, the composition is present in or on a live pest.

In some instances, the delivery vehicle is the food or water of the host. In other instances, the delivery vehicle is a food source for the host. In some instances, the delivery vehicle is a food bait for the host. In some instances, the composition is a comestible agent consumed by the host. In some instances, the composition is delivered by the host to a second host, and consumed by the second host. In some instances, the composition is consumed by the host or a second host, and the composition is released to the surrounding of the host or the second host via the waste (such as feces) of the host or the second host. In some instances, the defined bacterial co-culture composition is included in food bait intended to be consumed by a host or carried back to its colony.

In some instances, the defined bacterial co-culture may make up about 0.1% to about 100% of the composition, such as any one of about 0.01% to about 100%, about 1% to about 99.9%, about 0.1% to about 10%, about 1% to about 25%, about 10% to about 50%, about 50% to about 99%, or about 0.1% to about 90% of active ingredients (such as phage, lysin or bacteriocin). In some instances, the composition includes at least any of 0.1%, 0.5%, 1%, 5%, 10%, 20%, 30%, 40%, 50%, 60%, 70%, 80%, 90%, 95%, or more active ingredients (such as phage, lysin or bacteriocin). In some instances, the concentrated agents are preferred as commercial products, the final user normally uses diluted agents, which have a substantially lower concentration of active ingredient.

Liquid Formulations

The compositions provided herein may be in a liquid formulation. Liquid formulations are generally mixed with water, but in some instances may be used with crop oil, diesel fuel, kerosene or other light oil as a carrier. The amount of active ingredient often ranges from about 0.5 to about 80 percent by weight.

An emulsifiable concentrate formulation may contain a liquid active ingredient, one or more petroleum-based solvents, and an agent that allows the formulation to be mixed with water to form an emulsion. Such concentrates may be used in agricultural, ornamental and turf, forestry, structural, food processing, livestock, and public health pest formulations. These may be adaptable to application equipment from small portable sprayers to hydraulic sprayers, low-volume ground sprayers, mist blowers, and low-volume aircraft sprayers. Some active ingredients are readily dissolved in a liquid carrier. When mixed with a carrier, they form a solution that does not settle out or separate, e.g., a homogenous solution. Formulations of these types may include an active ingredient, a carrier, and one or more other ingredients. Solutions may be used in any type of sprayer, indoors and outdoors.

In some instances, the composition may be formulated as an invert emulsion. An invert emulsion is a water-soluble active ingredient dispersed in an oil carrier. Invert emulsions require an emulsifier that allows the active ingredient to be mixed with a large volume of petroleum-based carrier, usually fuel oil. Invert emulsions aid in reducing drift. With other formulations, some spray drift results when water droplets begin to evaporate before reaching target surfaces; as a result, the droplets become very small and lightweight. Because oil evaporates more slowly than water, invert emulsion droplets shrink less and more active ingredient reaches the target. Oil further helps to reduce runoff and improve rain resistance. It further serves as a sticker-spreader by improving surface coverage and absorption. Because droplets are relatively large and heavy, it is difficult to get thorough coverage on the undersides of foliage. Invert emulsions are most commonly used along rights-of-way where drift to susceptible non-target areas can be a problem.

A flowable or liquid formulation combines many of the characteristics of emulsifiable concentrates and wettable powders. Manufacturers use these formulations when the active ingredient is a solid that does not dissolve in either water or oil. The active ingredient, impregnated on a substance such as clay, is ground to a very fine powder. The powder is then suspended in a small amount of liquid. The resulting liquid product is quite thick. Flowables and liquids share many of the features of emulsifiable concentrates, and they have similar disadvantages. They require moderate agitation to keep them in suspension and leave visible residues, similar to those of wettable powders.

Flowables/liquids are easy to handle and apply. Because they are liquids, they are subject to spilling and splashing. They contain solid particles, so they contribute to abrasive wear of nozzles and pumps. Flowable and liquid suspensions settle out in their containers. Because flowable and liquid formulations tend to settle, packaging in containers of five gallons or less makes remixing easier.

Aerosol formulations contain one or more active ingredients and a solvent. Most aerosols contain a low percentage of active ingredients. There are two types of aerosol formulations—the ready-to-use type commonly available in pressurized sealed containers and those products used in electrical or gasoline-powered aerosol generators that release the formulation as a smoke or fog.

Ready to use aerosol formulations are usually small, self-contained units that release the formulation when the nozzle valve is triggered. The formulation is driven through a fine opening by an inert gas under pressure, creating fine droplets. These products are used in greenhouses, in small areas inside buildings, or in localized outdoor areas. Commercial models, which hold five to 5 pounds of active ingredient, are usually refillable.

Smoke or fog aerosol formulations are not under pressure. They are used in machines that break the liquid formulation into a fine mist or fog (aerosol) using a rapidly whirling disk or heated surface.

Dry or Solid Formulations

Dry formulations can be divided into two types: ready-to-use and concentrates that must be mixed with water to be applied as a spray. Most dust formulations are ready to use and contain a low percentage of active ingredients (less than about 10 percent by weight), plus a very fine, dry inert carrier made from talc, chalk, clay, nut hulls, or volcanic ash. The size of individual dust particles varies. A few dust formulations are concentrates and contain a high percentage of active ingredients. Mix these with dry inert carriers before applying. Dusts are always used dry and can easily drift to non-target sites.

In some instances, the composition is formulated as granules. Granular formulations are similar to dust formulations, except granular particles are larger and heavier. The coarse particles may be made from materials such as clay, corncobs, or walnut shells. The active ingredient either coats the outside of the granules or is absorbed into them. The amount of active ingredient may be relatively low, usually ranging from about 0.5 to about 15 percent by weight. Granular formulations are most often used to apply to the soil, insects or nematodes living in the soil, or absorption into plants through the roots. Granular formulations are sometimes applied by airplane or helicopter to minimize drift or to penetrate dense vegetation. Once applied, granules may release the active ingredient slowly. Some granules require soil moisture to release the active ingredient. Granular formulations also are used to control larval mosquitoes and other aquatic pests. Granules are used in agricultural, structural, ornamental, turf, aquatic, right-of-way, and public health (biting insect) pest-control operations.

In some instances, the composition is formulated as pellets. Most pellet formulations are very similar to granular formulations; the terms are used interchangeably. In a pellet formulation, however, all the particles are the same weight and shape. The uniformity of the particles allows use with precision application equipment.

In some instances, the composition is formulated as a powder. In some instances, the composition is formulated as a wettable powder. Wettable powders are dry, finely ground formulations that look like dusts. They usually must be mixed with water for application as a spray. A few products, however, may be applied either as a dust or as a wettable powder—the choice is left to the applicator. Wettable powders have about 1 to about 95 percent active ingredient by weight; in some cases more than about 50 percent. The particles do not dissolve in water. They settle out quickly unless constantly agitated to keep them suspended. They can be used for most pest problems and in most types of spray equipment where agitation is possible. Wettable powders have excellent residual activity. Because of their physical properties, most of the formulation remains on the surface of treated porous materials such as concrete, plaster, and untreated wood. In such cases, only the water penetrates the material.

In some instances, the composition is formulated as a soluble powder. Soluble powder formulations look like wettable powders. However, when mixed with water, soluble powders dissolve readily and form a true solution. After they are mixed thoroughly, no additional agitation is necessary. The amount of active ingredient in soluble powders ranges from about 15 to about 95 percent by weight; in some cases more than about 50 percent. Soluble powders have all the advantages of wettable powders and none of the disadvantages, except the inhalation hazard during mixing.

In some instances, the composition is formulated as a water-dispersible granule. Water-dispersible granules, also known as dry flowables, are like wettable powders, except instead of being dust-like, they are formulated as small, easily measured granules. Water-dispersible granules must be mixed with water to be applied. Once in water, the granules break apart into fineparticles similar to wettable powders. The formulation requires constant agitation to keep it suspended in water. The percentage of active ingredient is high, often as much as 90 percent by weight. Water-dispersible granules share many of the same advantages and disadvantages of wettable powders, except they are more easily measured and mixed. Because of low dust, they cause less inhalation hazard to the applicator during handling

In some instances, the composition includes a bait. The bait can be in any suitable form, such as a solid, paste, pellet or powdered form. The bait can also be carried away by the host back to a population of said host (e.g., a colony or hive). The bait can then act as a food source for other members of the colony.

The baits can be provided in a suitable “housing.” Such housings are commercially available and can be adapted to include the compositions described herein. The housing can be box-shaped for example, and can be provided in pre-formed condition or can be formed of foldable cardboard for example. Suitable materials for a housing include plastics and cardboard, particularly corrugated cardboard. The housing can contain a suitable trough inside which can hold the bait in place. A housing acts as a “feeding station” which provides the host with a preferred environment in which they can feed and feel safe from predators.

In some instances, the composition includes an attractant (e.g., a chemoattractant). The attractant may attract an adult host or immature host (e.g., larva) to the vicinity of the composition. Attractants include pheromones, a chemical that is secreted by an animal, especially an insect, which influences the behavior or development of others of the same species. Other attractants include sugar and protein hydrolysate syrups, yeasts, and rotting meat. Attractants also can be combined with an active ingredient and sprayed onto foliage or other items in the treatment area.

Various attractants are known which influence host behavior as a host's search for food, oviposition or mating sites, or mates. Attractants useful in the methods and compositions described herein include, for example, eugenol, phenethyl propionate, ethyl dimethylisobutyl-cyclopropane carboxylate, propyl benszodioxancarboxylate, cis-7,8-epoxy-2-methyloctadecane, trans-8,trans-0-dodecadienol, cis-9-tetradecenal (with cis-11-hexadecenal), trans-11-tetradecenal, cis-11-hexadecenal, (Z)-11,12-hexadecadienal, cis-7-dodecenyl acetate, cis-8-dodecenyulacetate, cis-9-dodecenyl acetate, cis-9-tetradecenyl acetate, cis-11-tetradecenyl acetate, trans-11-tetradecenyl acetate (with cis-11), cis-9,trans-11-tetradecadienyl acetate (with cis-9,trans-12), cis-9,trans-12-tetradecadienyl acetate, cis-7,cis-11-hexadecadienyl acetate (with cis-7,trans-11), cis-3,cis-13-octadecadienyl acetate, trans-3,cis-13-octadecadienyl acetate, anethole and isoamyl salicylate.

Adjuvants

In some instances, the composition provided herein may include an adjuvant. Adjuvants are chemicals that do not possess activity. Adjuvants are either pre-mixed in the formulation or added to the spray tank to improve mixing or application or to enhance performance. Adjuvants can be used to customize the formulation to specific needs and compensate for local conditions. Adjuvants may be designed to perform specific functions, including wetting, spreading, sticking, reducing evaporation, reducing volatilization, buffering, emulsifying, dispersing, reducing spray drift, and reducing foaming. Among nonlimiting examples of adjuvants included in the formulation are binders, dispersants and stabilizers, specifically, for example, casein, gelatin, polysaccharides (e.g., starch, gum arabic, cellulose derivatives, alginic acid, etc.), lignin derivatives, bentonite, sugars, synthetic water-soluble polymers (e.g., polyvinyl alcohol, polyvinylpyrrolidone, polyacrylic acid, etc.), PAP (acidic isopropyl phosphate), BHT (2,6-di-t-butyl-4-methylphenol), BHA (a mixture of 2-t-butyl-4-methoxyphenol and 3-t-butyl-4-methoxyphenol), vegetable oils, mineral oils, fatty acids and fatty acid esters.

Surfactants

In some instances, the composition provided herein includes a surfactant. Surfactants, also called wetting agents and spreaders, physically alter the surface tension of a spray droplet. Surfactants enlarge the area of formulation coverage, thereby increasing the host's exposure to the compositions of the invention. Surfactants are particularly important when applying a formulation to waxy or hairy surfaces. Without proper wetting and spreading, spray droplets often run off or fail to cover surfaces adequately. Among nonlimiting examples of surfactants included in the compositions described herein are alkyl sulfate ester salts, alkyl sulfonates, alkyl aryl sulfonates, alkyl aryl ethers and polyoxyethylenated products thereof, polyethylene glycol ethers, polyvalent alcohol esters and sugar alcohol derivatives.

Delivery

A host described herein can be exposed to any of the compositions described herein in any suitable manner that permits delivering or administering the composition to the insect. The defined bacterial co-culture compositions may be delivered either alone or in combination with other active or inactive substances and may be applied by, for example, spraying, microinjection, through plants, pouring, dipping, in the form of concentrated liquids, gels, solutions, suspensions, sprays, powders, pellets, briquettes, bricks and the like, formulated to deliver an effective concentration of the defined bacterial co-culture composition.

Amounts and locations for application of the compositions described herein are generally determined by the habits of the host, the lifecycle stage at which the microorganisms of the host can be targeted by the defined bacterial co-culture compositions, the site where the application is to be made, and the physical and functional characteristics of the defined bacterial co-culture compositions. In some embodiments, the defined bacterial co-culture composition described herein may be administered to the insect by oral ingestion.

In some instances, the insect can be simply “soaked” or “sprayed” with a solution including the defined bacterial co-culture composition. Alternatively, the defined bacterial co-culture compositions can be incorporated into to a food component (e.g., comestible) of the insect for ease of delivery and/or in order to increase uptake of the defined bacterial co-culture compositions by the insect. Methods for oral introduction include, for example, directly mixing a defined bacterial co-culture compositions with the insects food or spraying defined bacterial co-culture compositions in the insect's habitat or field. In some instances, for example, the defined bacterial co-culture compositions can be incorporated into, or overlaid on the top of, the insect's diet. For example, the defined bacterial co-culture compositions composition can be sprayed onto a field of crops which an insect inhabits.

The defined bacterial co-culture compositions can also be incorporated into the medium in which the insect grows, lives, reproduces, feeds, or infests. For example, a defined bacterial co-culture composition can be incorporated into a food container, feeding station, protective wrapping, or a hive. For some applications the defined bacterial co-culture composition may be bound to a solid support for application in powder form or in a “feeding station.” For example, in instances where the host is a honeybee, the compositions described herein can be administered by delivering the composition to a honeybee hive or at least one habitat where a honeybee grows, lives, reproduces, or feeds.

### Methods of Generating a Bacterial Co-Culture

In one embodiment, the invention provides methods of generating a defined bacterial co-culture. As used herein a “bacterial co-culture” refers to a bacterial cell culture, which includes at least the two bacterial strains of the present invention, described hereinabove.

The isolation, identification and culturing of the bacterial strains of the present invention (i.e., comprising at least two bacterial strains selected from S. alvi, G. apicola, G. apis, Bartonella apis, Lactobacillus spp., and Bifidobacterium spp.) can be effected using standard microbiological techniques. Examples of such techniques may be found in Gerhardt, P. (ed.) Methods for General and Molecular Microbiology. American Society for Microbiology, Washington, D.C. (1994) and Lennette, E. H. (ed.) Manual of Clinical Microbiology, Third Edition. American Society for Microbiology, Washington, D.C. (1980).

In one embodiment, isolation is effected by streaking a specimen on a solid medium (e.g., nutrient agar plates) to obtain single colonies and to reduce the likelihood of working with a culture which has become contaminated and/or has accumulated mutations. In one embodiment, the defined bacterial co-culture of the invention is grown on blood-columbia (B-COL) agar.

In one embodiment, the bacterial strains of the present invention can be propagated in a liquid medium under aerobic, micro-aerophilic or anaerobic conditions.

Medium for growing the bacterial strains of the present invention includes a carbon source, a nitrogen source and inorganic salts as well as specially required substances such as vitamins, amino acids, nucleic acids and the like.

Examples of suitable carbon sources which can be used for growing the bacterial strains of the present invention include, but are not limited to, starch, peptone, yeast extract, amino acids, sugars such as glucose, arabinose, mannose, glucosamine, maltose, and the like; salts of organic acids such as acetic acid, fumaric acid, adipic acid, propionic acid, citric acid, gluconic acid, malic acid, pyruvic acid, malonic acid and the like; alcohols such as ethanol and glycerol and the like; oil or fat such as soybean oil, rice bran oil, olive oil, corn oil, sesame oil. The amount of the carbon source added varies according to the kind of carbon source and is typically between 1 to 100 gram per liter medium. Preferably, glucose, starch, and/or peptone is contained in the medium as a major carbon source, at a concentration of 0.1-5% (W/V).

Examples of suitable nitrogen sources which can be used for growing the bacterial strains of the present invention include, but are not limited to, amino acids, yeast extract, tryptone, beef extract, peptone, potassium nitrate, ammonium nitrate, ammonium chloride, ammonium sulfate, ammonium phosphate, ammonia or combinations thereof. The amount of nitrogen source varies according the nitrogen source, typically between 0.1 to 30 gram per liter medium.

As the inorganic salts, potassium dihydrogen phosphate, dipotassium hydrogen phosphate, disodium hydrogen phosphate, magnesium sulfate, magnesium chloride, ferric sulfate, ferrous sulfate, ferric chloride, ferrous chloride, manganous sulfate, manganous chloride, zinc sulfate, zinc chloride, cupric sulfate, calcium chloride, sodium chloride, calcium carbonate, sodium carbonate can be used alone or in combination. The amount of inorganic acid varies according to the kind of the inorganic salt, typically between 0.001 to 10 gram per liter medium.

Examples of specially required substances include, but are not limited to, vitamins, nucleic acids, yeast extract, peptone, meat extract, malt extract, dried yeast and combinations thereof.

Cultivation is effected at a temperature, which allows the growth of the probiotic bacterial strains of the present invention, essentially, between 28° C. and 46° C. A preferred temperature range is 30-37° C.

For optimal growth, the medium is preferably adjusted to pH 7.0-7.4.

It will be appreciated that cultivation time may differ depending on the type of culture medium used and the concentration of sugar as a major carbon source. Typically, cultivation lasts between 24-96 hours to reach 80% sporulation of cultures.

Cultured bacterial cells can be collected using methods which are well known in the art. Examples include, but are not limited to, membrane filtration and centrifugal separation.

The pH may be adjusted using sodium hydroxide and the like and the culture may be dried using a freeze dryer, until the water content becomes equal to 4% or less.

In one embodiment, each bacterial strain is cultured individually for a period of time before being included in a co-culture. In one embodiment, at least two bacterial strains selected from S. alvi, G. apicola, G. apis, Bartonella apis, Lactobacillus spp., and Bifidobacterium spp. are cultured separately for a time period of at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 16 hours, at least 24 hours, for at least 36 hours, for at least 48 hours, for at least 60 hours for at least 72 hours, for at least 84 hours, for at least 96 hours or for more than 96 hours prior to being combined into a single culture.

In one embodiment, the defined bacterial co-culture described above, may be obtained by propagating each strain together as a single culture. Thus, in one embodiment, at least two bacterial stains selected from S. alvi, G. apicola, G. apis, Bartonella apis, Lactobacillus spp., and Bifidobacterium spp. are cultured together for a time period of at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 16 hours, at least 24 hours, for at least 36 hours, for at least 48 hours, for at least 60 hours for at least 72 hours, for at least 84 hours, for at least 96 hours or for more than 96 hours prior to being collected.

In one embodiment, the final concentration of each bacterial strain in the defined co-culture is between about 10⁴ to 10¹⁰ organisms/ml. However, one of ordinary skill in the art will appreciate that this ratio may vary depending upon the culture medium used, the relative ages of the cultures and their viability.

### Methods of Use

In one embodiment, the invention provides methods of using the defined bacterial co-culture of the invention to prevent a disease or disorder or to promote core microbiome development in bees. Core microbiome development can be promoted by providing and effective amount of the defined bacterial co-culture of the invention as a probiotic to a bee or bee colony. An effective amount of the defined bacterial co-culture of the invention described herein is an amount that achieves a desired result (e.g., improved growth of core microbiome) in the bees or bee colony. An effective amount can be provided in a single feeding or application, or over time. An effective amount can depend on several factors, such as colony size, method of feeding, and desired effect. An effective amount necessary to achieve a desired result can be determined or modified by one of skill in the art.

In some embodiments, the composition is effective to increase health and/or survival of the host. In some embodiments, the composition is effective to increase host fitness, increase host lifespan, increase effective pollination, increase generation of a host product, increase host reproduction, or a combination thereof.

Exemplary diseases and disorders that can be prevented using the defined bacterial co-culture of the invention include, but are not limited to, colony collapse disorder, infection by a viral pathogen, infection by a bacterial pathogen, Deformed Wing Virus (DWV) infection, opportunistic bacterial infection of adult worker bees (e.g., such as infections by S. marcescens and other Enterobacteriaceae pathogens), and also including infection by protozoan parasites such as Nosema species or Crithidia species. The invention may also protect against larval disease, including fungal pathogens such as chalkbrood and bacterial disease such as American Foulbrood (AFB) disease.

In some embodiments, the compositions disclosed herein may be used to increase the fitness of a bee host. The increase in fitness may arise from an alteration in the microorganisms resident in the host, wherein the alterations are a consequence of administration of a defined bacterial co-culture comprising at least at least two bacterial strains native to the bee gut and have beneficial or advantageous effects on the host.

In some instances, the increase in host fitness may manifest as an improvement in the physiology of the host (e.g., improved health or survival) as a consequence of administration of a defined bacterial co-culture composition. In some instances, the fitness of an organism may be measured by one or more parameters, including, but not limited to, reproductive rate, lifespan, mobility, fecundity, body weight, metabolic rate or activity, or survival in comparison to a host organism to which the defined bacterial co-culture composition has not been administered. For example, the methods or compositions provided herein may be effective to improve the overall health of the host or to improve the overall survival of the host in comparison to a host organism to which the defined bacterial co-culture composition has not been administered. In some instances, the improved survival of the host is about 2%, 5%, 10%, 20%, 30%, 40%, 50%, 60%, 70%, 80%, 90%, 100%, or greater than 100% greater relative to a reference level (e.g., a level found in a host that does not receive a defined bacterial co-culture). In some instances, the methods and compositions are effective to increase host reproduction (e.g., reproductive rate) in comparison to a host organism to which the defined bacterial co-culture composition has not been administered. In some instances, the methods and compositions are effective to increase other physiological parameters, such as mobility, body weight, life span, fecundity, or metabolic rate, by about 2%, 5%, 10%, 20%, 30%, 40%, 50%, 60%, 70%, 80%, 90%, 100%, or greater than 100% relative to a reference level (e.g., a level found in a host that does not receive a defined bacterial co-culture).

In some instances, the increase in host fitness may manifest as an increased production of a product generated by said host in comparison to a host organism to which the defined bacterial co-culture composition has not been administered. In some instances, the methods or compositions provided herein may be effective to increase the production of a product generated by the host, as described herein (e.g., honey, beeswax, beebread), by about 2%, 5%, 10%, 20%, 30%, 40%, 50%, 60%, 70%, 80%, 90%, 100%, or greater than 100% relative to a reference level (e.g., a level found in a host that does not receive a defined bacterial co-culture).

In some instances, the increase in host fitness may manifest as an increase in the frequency or efficacy of a desired activity carried out by the host (e.g., pollination) in comparison to a host organism to which the defined bacterial co-culture composition has not been administered. In some instances, the methods or compositions provided herein may be effective to increase the frequency or efficacy of a desired activity carried out by the host by about 2%, 5%, 10%, 20%, 30%, 40%, 50%, 60%, 70%, 80%, 90%, 100%, or greater than 100% relative to a reference level (e.g., a level found in a host that does not receive a defined bacterial co-culture).

In some embodiments, the methods or compositions provided herein may be effective to increase the host's resistance to parasites or pathogens (e.g., fungal, bacterial, or viral pathogens; or parasitic mites (e.g., Varroa destructor mite in honeybees)) in comparison to a host organism to which the defined bacterial co-culture has not been administered. In some instances, the methods or compositions provided herein may be effective to increase the host's resistance to a pathogen or parasite (e.g., fungal, bacterial, or viral pathogens; or parasitic mites (e.g., Varroa destructor mite in honeybees)) by about 2%, 5%, 10%, 20%, 30%, 40%, 50%, 60%, 70%, 80%, 90%, 100%, or greater than 100% relative to a reference level (e.g., a level found in a host that does not receive a defined bacterial co-culture).

Host fitness may be evaluated using any standard methods in the art. In some instances, host fitness may be evaluated by assessing an individual host. Alternatively, host fitness may be evaluated by assessing a host population. For example, an increase in host fitness may manifest as an increase in successful competition against other insects, thereby leading to an increase in the size of the host population.

Typical concentration range of probiotic microorganisms administered, may be 10³ to 10¹³ cells per day. In various embodiments, at least about 10⁴, at least about 10⁵, at least about 10⁶, or more than 10⁶ cells per day are used in probiotic administration. However, it will be appreciated that the amount of bacteria to be administered will vary according to a number of parameters including the size of a bee colony.

Compositions described herein can be provided to a bee or bee colony. This can be done via feeding, wherein an effective amount of the composition is placed in or near a bee colony's hive so that the bees can feed on the composition. Methods for feeding bees are well known in the art, and include, for example, utilizing a frame feeder, a simple shallow tray, a bag feeder, or ajar feeder. Where the composition comprises a gel-based carrier, or is formulated as a syrup, the composition can be applied directly one or more of the frames of the colony's hive. Application to the frames of the hive allows nurse bees to have direct access to the probiotic composition.

In principle, every feed can be used that is accepted by the bee to be fed. This includes any kind material that is consumed orally by the bees, independent on whether it is natural feed, agricultural feed or laboratory feed and independent on whether it is consumed naturally or is administered by means of technical devices or is taken up casually. In one embodiment, the feed that is used to induce the production of the gene encoded molecules in the bees is either a liquid feed comprising the defined bacterial co-culture, a dry feed mixed with a solution comprising the defined bacterial co-culture or a dry feed comprising the defined bacterial co-culture in any of these formulations.

As detailed herein, bee feeding is common practice amongst bee-keepers, for providing both nutritional and other, for example, supplemental needs. Bees typically feed on honey and pollen, but have been known to ingest non-natural feeds as well. Bees can be fed various foodstuffs including, but not limited to Wheast (a dairy yeast grown on cottage cheese), soybean flour, yeast (e.g. brewer's yeast, torula yeast) and yeast products products-fed singly or in combination and soybean flour fed as a dry mix or moist cake inside the hive or as a dry mix in open feeders outside the hive. Also useful is sugar, or a sugar syrup. The addition of 10 to 12 percent pollen to a supplement fed to bees improves palatability. The addition of 25 to 30 percent pollen improves the quality and quantity of essential nutrients that are required by bees for vital activity.

Cane or beet sugar, isomerized corn syrup, and type-50 sugar syrup are satisfactory substitutes for honey in the natural diet of honey bees. The last two can be supplied only as a liquid to bees.

Liquid feed can be supplied to bees inside the hive by, for example, any of the following methods: friction-top pail, combs within the brood chamber, division board feeder, boardman feeder, etc. Dry sugar may be fed by placing a pound or two on the inverted inner cover. A supply of water must be available to bees at all times. In one embodiment, pan or trays in which floating supports-such as wood chips, cork, or plastic sponge-are present are envisaged. Detailed descriptions of supplemental feeds for bees can be found in, for example, USDA publication by Standifer, et al 1977, entitled “Supplemental Feeding of Honey Bee Colonies” (USDA, Agriculture Information Bulletin No. 413).

All the bees in a hive are potentially susceptible to the pathogenic diseases detailed herein. Thus, according to some embodiments, the bees can be nurse bees, forager bees, hive bees, guard bees and the like.

Also provided is a method for reducing the susceptibility of a bee to a disease caused by pathogens, the method effected by feeding the bee on an effective amount of a defined bacterial co-culture. Methods for reducing the susceptibility of a bee colony or bee-hive to bee pathogens by feeding defined bacterial co-culture are envisaged. Thus, in some embodiments, the present invention can be used to benefit any numbers of bees, from a few in the hive, to the entire bee population within a hive and its surrounding area. It will be appreciated, that in addition to feeding of defined bacterial co-culture for reduction of the bee pathogen infection and infestation, enforcement of proper sanitation (for example, refraining from reuse of infested hives) can augment the effectiveness of treatment and prevention of infections.

Antibiotics

A composition comprising the defined bacterial co-culture of the present invention may be administered in combination with a therapeutically-effective amount of an antibiotic. For example, the compositions of the present invention may be administered in combination with a therapeutically-effective amount of lincomycin, oxytetracycline, tylosine tartarate, fumagillin, amitraz, oxalic acid, thymol, or natural plant-derived compounds or mixtures of compounds. In various embodiments, the composition comprising the defined bacterial co-culture of the present invention can be administered prior to, subsequent to, or concurrently with a therapeutically-effective amount of an antibiotic.

### Kits

The invention also includes a kit comprising a defined bacterial co-culture of the invention. In one embodiment, the kit may also comprise instructional material which describes, for instance, methods of propagating a defined bacterial co-culture, or methods of administering a defined bacterial co-culture of the invention to a target bee or bee colony.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Use of Probiotics to Improve Bee Health

Here data supporting two claims regarding the use of probiotics to improve bee health is presented. The data demonstrate that in vitro co-culture of a probiotic cocktail bacteria prior to inoculation increases the regularity of colonization. This is in contrast to the usual method of inoculating multiple species which involves separate culture prior to inoculation. The data further demonstrate that a defined probiotic community of bacteria helps bees with a dysbiotic gut microbiome resist infection by the opportunistic pathogen Serratia marcescens. While many “probiotics for bees” are currently available, they generally do not consist of microbes isolated from the bee gut, and have not been shown to have any quantifiable protective effect or to achieve stable colonization of bee guts following ingestion.

The methods used in these experiments are now described

Bacterial Culture

Strains of Snodgrassella alvi, Gilliamella apicola, Bartonella apis, Bifidobacterium asteroides, and Firmicutes (Table 1) were grown on blood-columbia (B-COL) agar in a 5% CO₂ incubator for 48-72 hours.

TABLE 1
Bee gut strains tested in probiotic co-culture

| | | What other | Member |
|---|---|---|---|
| | | strains are | of current |
| | Culturing | in stable | probiotic |
| | method | co-culture | bacterial |
| Strain | established | (ID #s) | cocktail (Y/N) |
| Snodgrassella alvi wkB2 | Y | 2, 3, 4, 5, 6, 7 | Y |
| Gillamella apicola wkB1 | Y | 1, 3, 4, 5, 6, 7 | Y |
| Gillamella apicola wkB7 | Y | 1, 2, 4, 5, 6, 7 | Y |
| Bartonella apis PEB0150 | Y | 1, 2, 3, 5, 6, 7 | N |
| Lactobacillus “Firm-5” | Y | 1, 2, 3, 4, 6, 7 | Y |
| wk610 | | | |
| Lactobacillus “Firm-5” | Y | 1, 2, 3, 4, 5, 7 | Y |
| wkB8 | | | |
| Bifidobacterium | | | |
| asteroides | | | |
| LCep5 | Y | 1, 2, 3, 4, 5, 6 | Y |

Defined Bacterial Communities

After ˜72 hours individual in vitro culture growth, equal optical density (OD) ratios of strains were mixed together to a volume of 200 μL and spot plated on a single B-COL plate. After 48 hours, the resulting mix was then scraped into a 1.5 mL microcentrifuge tube, washed in PBS, and resuspended in 10% glycerol before freezing at −80° C. Defined communities described in this work were of two compositions. The first composition (to test the effect of separate culture vs co-culture) was composed of Snodgrassella alvi wkB2, Gilliamella apicola wkB1, Gilliamella apicola wkB7, Bartonella apis PEB0150, Lactobacillus “Firm-5” wkB10, and Lactobacillus “Firm-5” wkB8. Strains in the 2nd composition (to test health effects of probiotic supplementation) included Snodgrassella alvi wkB2, Gilliamella apicola wkB1, Gilliamella apicola wkB7, Lactobacillus “Firm-5” wkB10, and Lactobacillus “Firm-5” wkB8.

Inoculating with Bacterial Communities

To inoculate bees, a suspension containing the defined community solution was applied directly onto pollen feed. Briefly, frozen aliquots of defined communities were thawed and diluted to an OD of 0.2, and 200 μL of this solution was combined with a 50% sucrose in water solution. Approximately 1 mL of this solution was used to inoculated ˜35 bees in each single cup.

The results of the experiments are now described

Pre-Inoculation Co-Culture of Bacteria Increases Stability and Uniformity of Probiotic Inoculation.

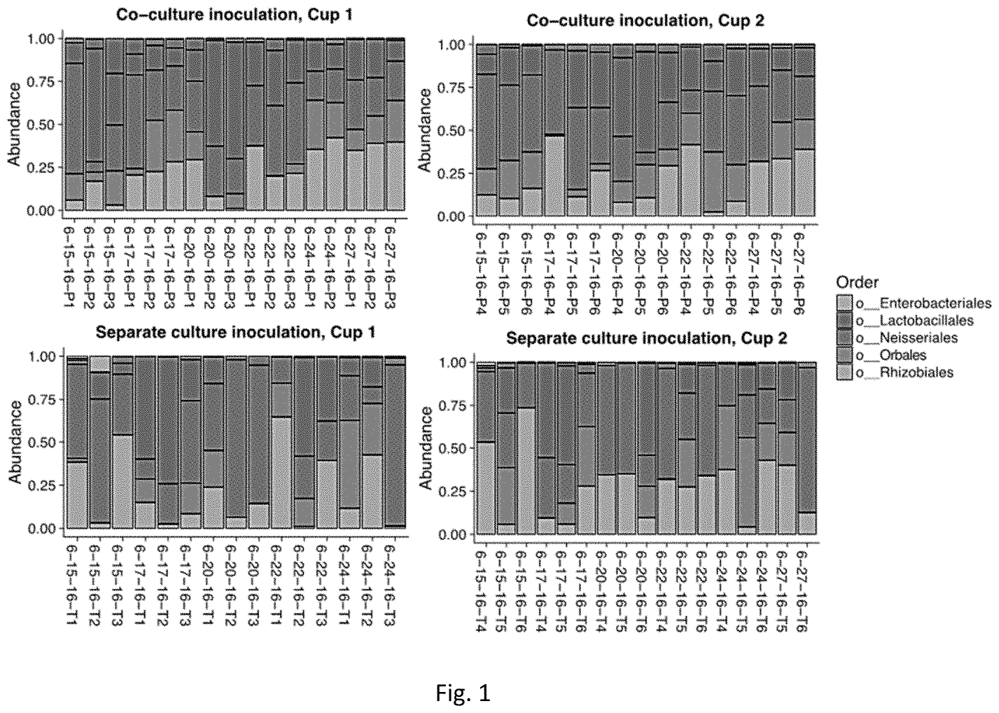

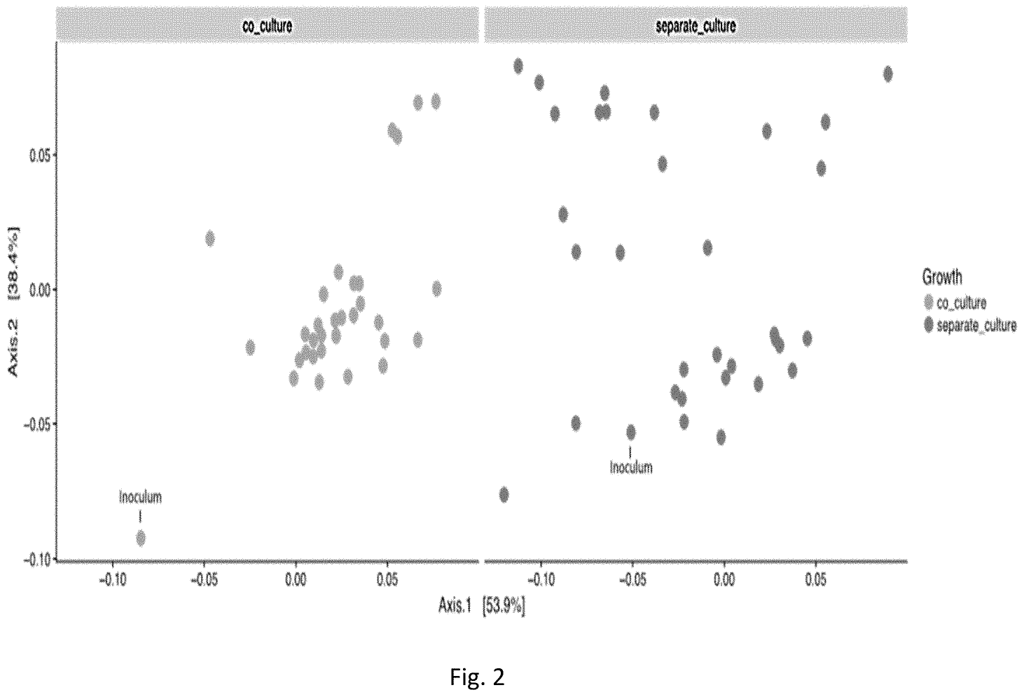

A probiotic culture was grown under two different conditions: (1) “separate culture”, where each bacterial species was cultured individually and mixed immediately prior to inoculation and (2) “co-culture” where bacteria were pooled together and grown overnight before inoculation into bees. Bees were sampled regularly over 12 days to assess the composition and assembly of the gut community. FIG. 1 shows the relative abundance of bacteria in colonized bees. FIG. 2 shows that co-culture inoculated bee gut microbiomes are more similar than separate-culture inoculated bees.

Defined Community Recapitulates Bee Weight Gain from Normal Bee Gut Bacteria

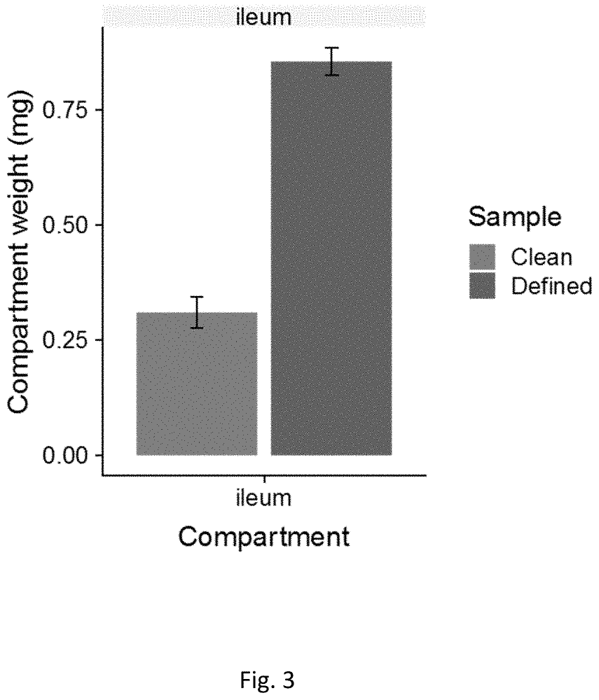

Bees were isolated from a single hive, and then either kept germ-free (“Clean”) or inoculated with the co-cultured defined community (“Defined”). After 7 days, bees were dissected and individual gut compartments weighed and measured. n=13-14 bees per condition. FIG. 3 shows that the defined community (probiotic) causes increased ileum weight, similar to the increased ileum weight previously shown to result from colonization by the complete gut community.

Defined Community Recapitulates Changes in Gene Expression Associated with Normal Bee Gut Bacteria

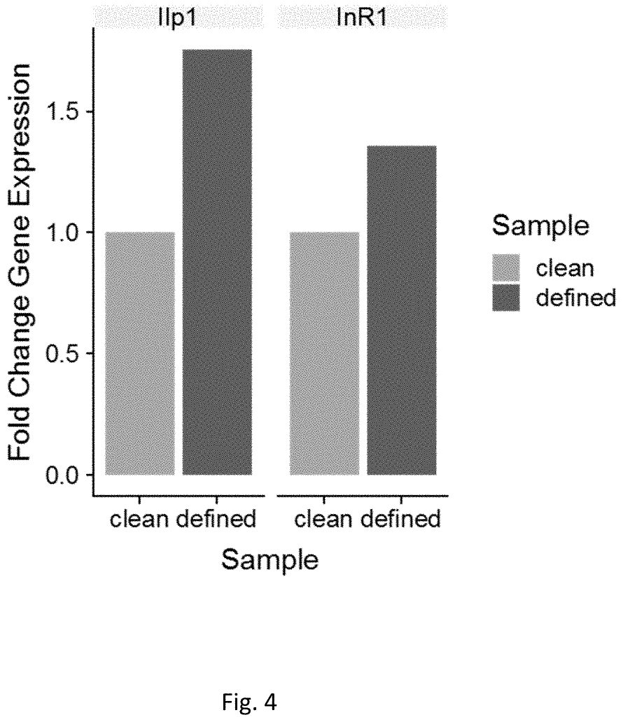

Bees were isolated from a single hive, and then either kept germ-free (“Clean”) or inoculated with the co-cultured defined community (“Defined”). After 7 days, bees were dissected and RNA isolated from whole abdomens. cDNA was transcribed, and then quantitative RT-PCR was performed. N=7-8 bees per condition. Ilp1 and InR1 are two insulin/insulin-like signaling genes that were previously shown to be upregulated by the complete gut community. FIG. 4 shows that the defined community (probiotic) causes increased gene expression of insulin-signaling genes, similar to the increased gene expression by the complete gut community.

Inoculation with Defined Probiotic Community Reduces Mortality of Gut-Dysbiotic Bees Exposed to the Pathogen Serratia.

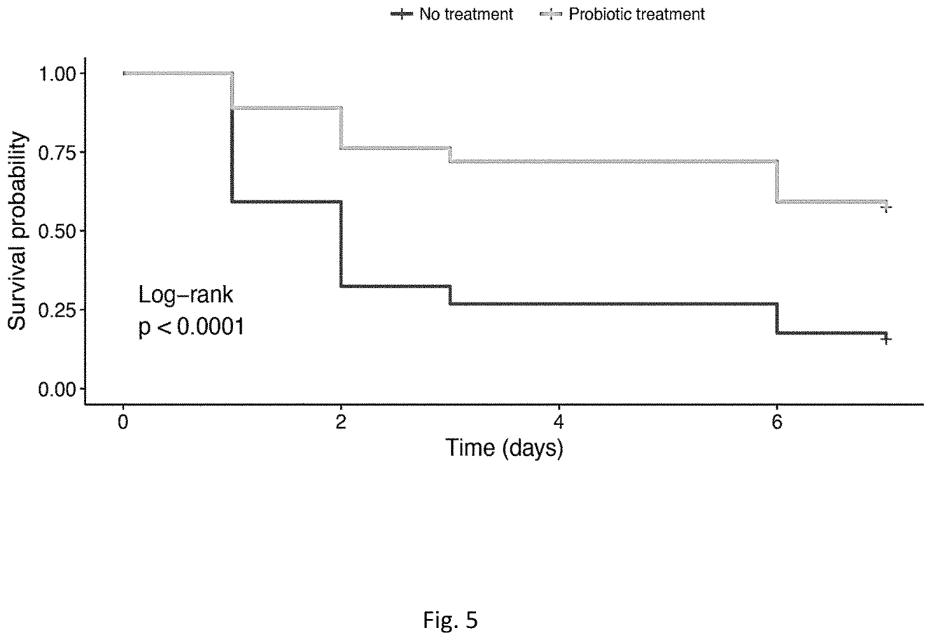

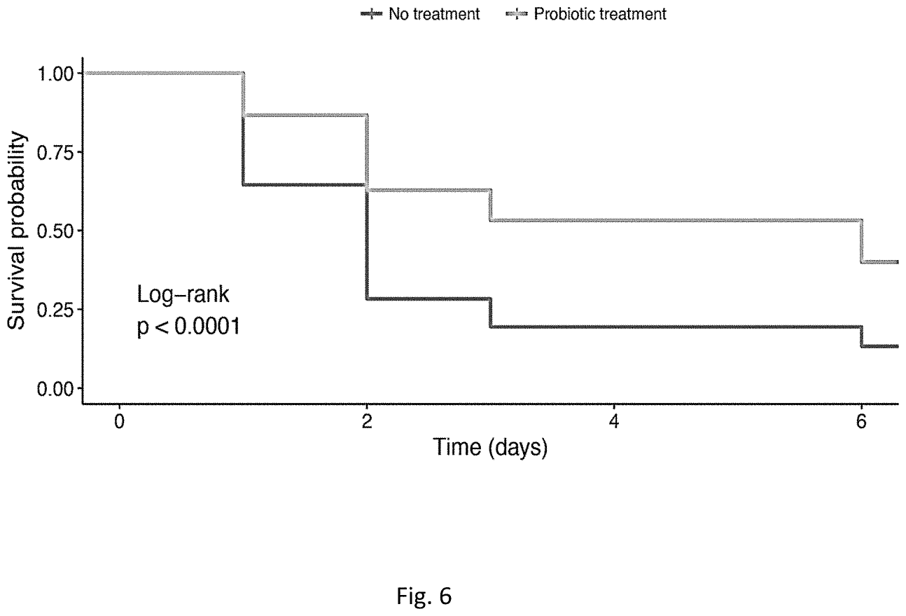

Previously, it has been shown that antibiotic treatment of honey bees increases susceptibility to the bacterial pathogen Serratia, likely due to a disrupted gut microbiome. Here, the ability of administration of a defined probiotic cocktail after antibiotic perturbation (5 days of oxytetracycline treatment at 450 ug/ml), such as bees may experience when hives are treated with antibiotics, to reduce this mortality was tested. FIG. 5 and FIG. 6 show survival of bees after exposure to low or high doses of Serratia, in the presence or absence of probiotic treatment.

Treatment with Lower Concentrations of Antibiotic for Less Time and with Other Antibiotics

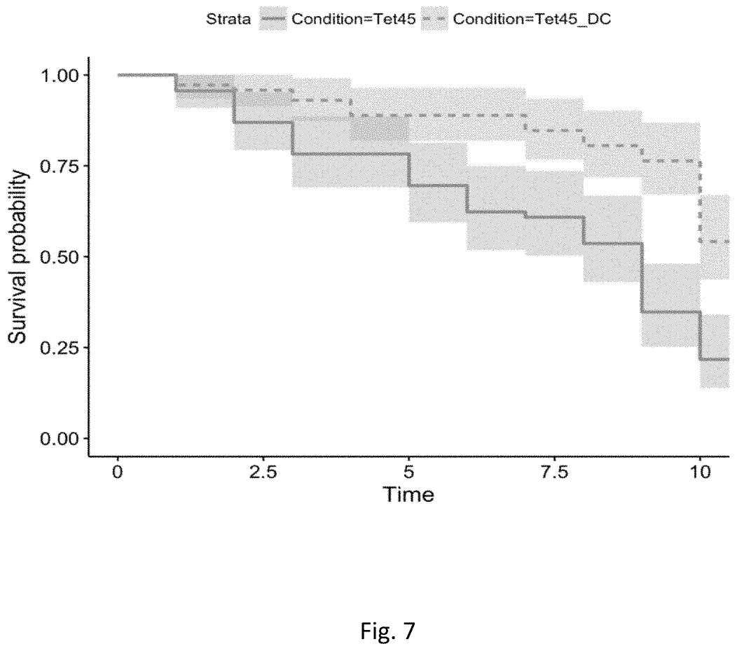

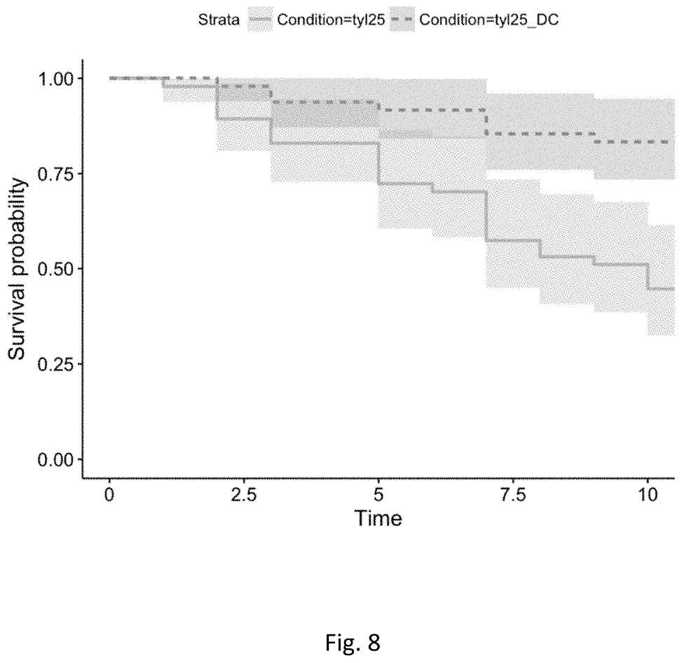

Previous studies have demonstrated that inoculation of bees using a probiotic mixture of bacteria after treatment with a continuous high dose of oxytetracycline (5 days at 450 μg/ml). FIG. 7 and FIG. 8 demonstrate the mortality of 5 day old bees collected from hives and treated for three days with an acute dose of either oxytetracycline (45 μg/ml) or tylosine tartarate (25 μg/ml) had better survival metrics when treated with the defined community probiotic prior to oral pathogenic bacterial exposure (5 μl of OD₆₀₀=1 S. marcescens strain N10A28).

The Protective Benefit of the Probiotic Cocktail Showed Benefit in Survivability After Hive Treatment with an Antibiotic Regime.

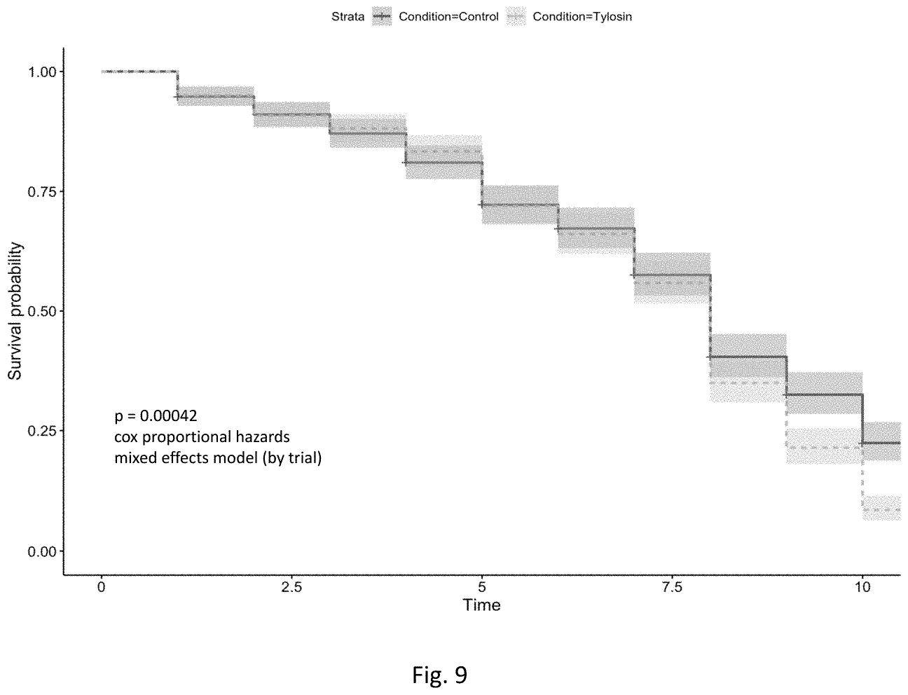

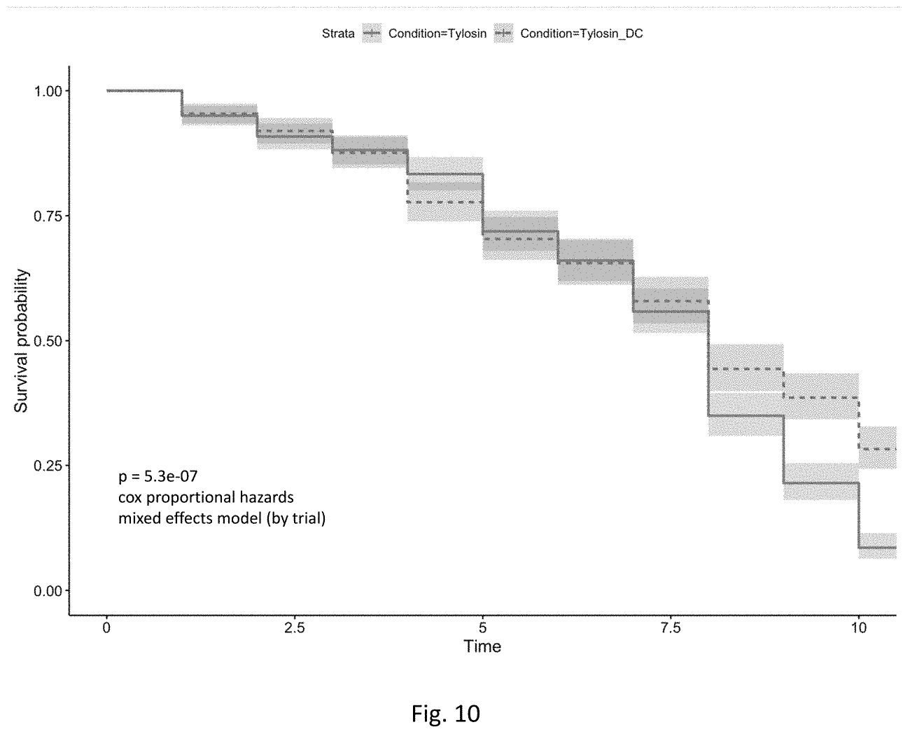

Four batches of individual paired hives were treated (experimental group) or not (control group with a tylosin tartarate treatment regime. This included 3 weeks of 200 mg antibiotic in 20 g powdered sugar, dusted over frames every 7 days. Five days after the final treatment bees were brought back to the lab and half were given the probiotic cocktail. Two days after this, each group was split in half—one half to act as control groups and the other half to be fed sugar water suspensions of Serratia N10A28. All conditions were housed in 3 cup cages of 40 bees each. Thus, there were 4 conditions examined for both the control hives and the tylosin treated hives. Bacterial suspensions were replaced every 3 days and mortality was assessed daily for 10 days. FIG. 9 demonstrates that, for control and antibiotic treated hives with no probiotic, Tylosin treated hives had bees with lower survival after bacterial challenge than did bees from control hives. FIG. 10 demonstrates that for control and antibiotic treated hives with and without probiotic, treatment of bees with probiotic mixture after antibiotic treatment increased survival significantly.

Bees Treated with Probiotic Mix Exhibited Pronounced Upregulation of Immunity Related Genes within Hours of Treatment

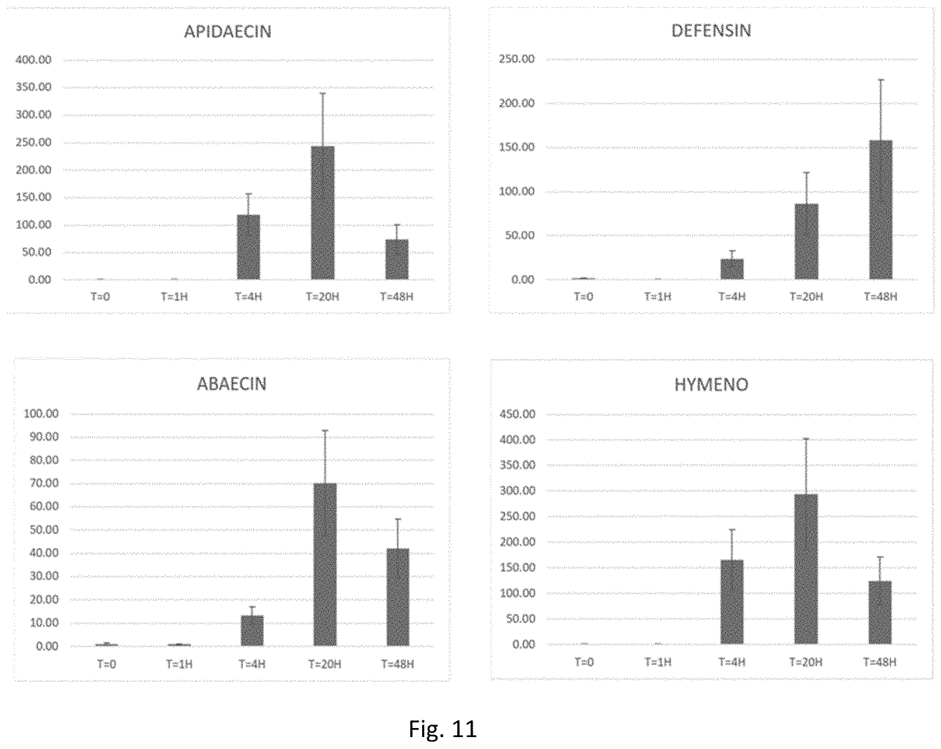

One day old germ free bees were fed 3 μl of probiotic mixture. Samples were taken prior to treatment and at 4, 20, and 48 hours after treatment. RNA was extracted and expression of antimicrobial peptide (AMP) genes was assessed relative to a housekeeping gene (RPSS). FIG. 11 shows 5 replicates at each time point and their fold expression relative to the pre-treatment samples. AMP genes were observed to be upregulated within 4 hours of treatment and this continued through the subsequent samplings.

Bees Colonized with Specific Probiotic Isolates Demonstrated Significantly Higher Survival Rate Against a Pathogen (Serratia Strain N10A28)

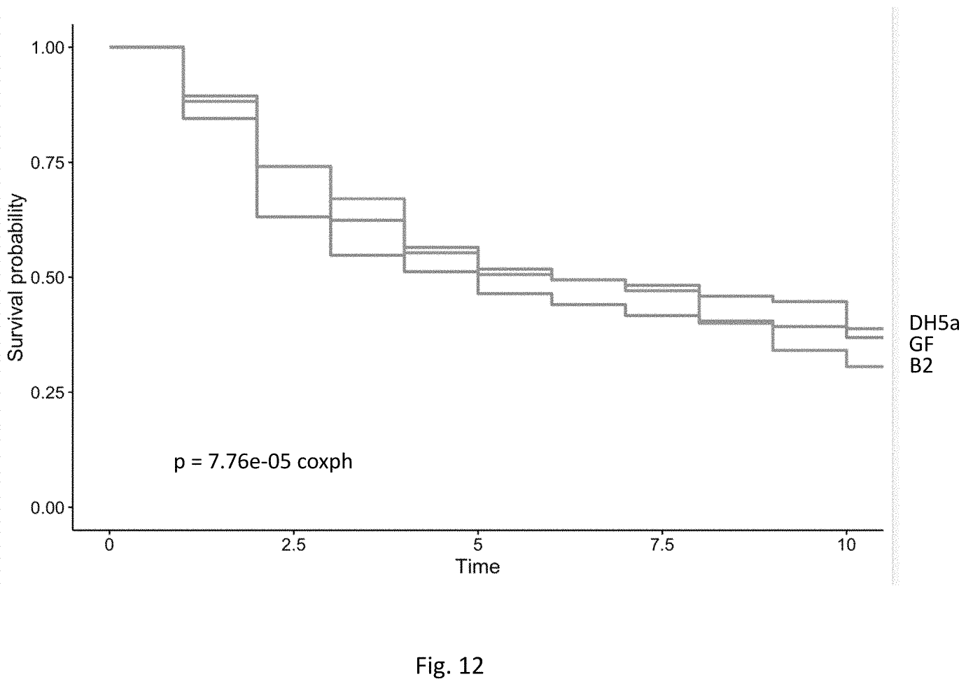

Germ free bees were inoculated with mono or dual BGM isolates at age=1-2 day and placed in cups (n=6-7 per cup, 2-3 cups/inoculum). At age=3-4 days, bees were fed 5 μl of Serratia marcescens N10A28 at OD₆₀₀=2. Mortality was recorded for 10 days. Inoculations included: None=GF &/or nonspecific bacteria (DH5a); Snod (B2); Bifido (LC5); B2+LC5; Firm-5 (wkB8 and wkB10); and Defined Community (DC). DH5a, B2 and B2+LC5 were equivalent to germ free (GF) (FIG. 12). The Firm-5 had notably lower mortality rates.

Bees Colonized with Specific Combinations of Probiotic Isolates Demonstrate Significantly Lower Infection Levels After Infection with the Pathogen (Serratia Strain KZ11,“SnM”)

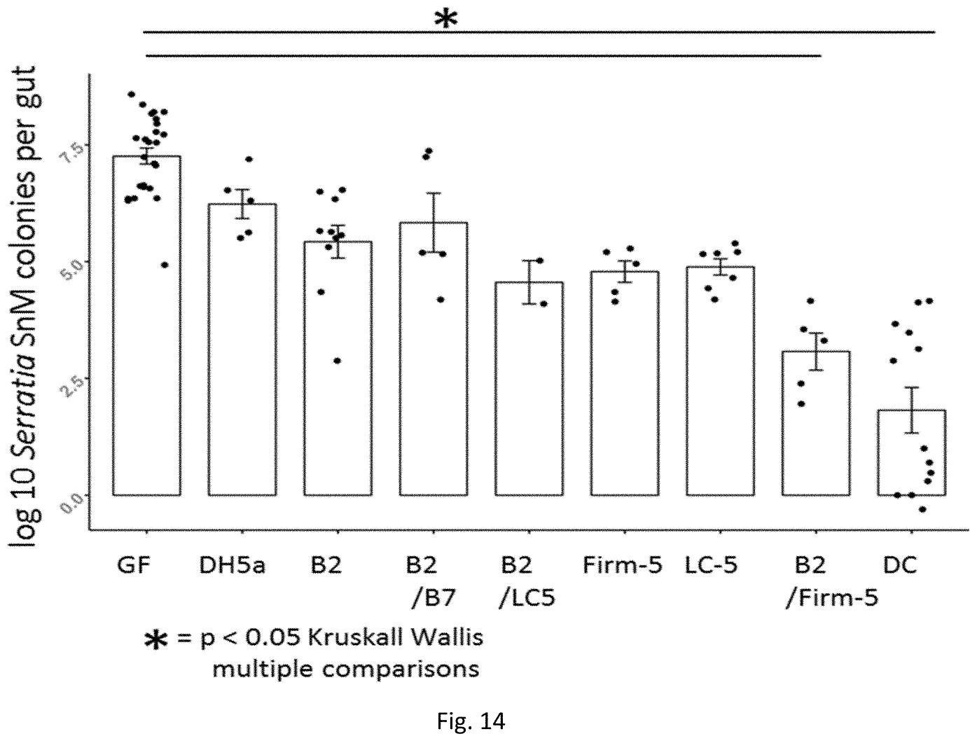

Germ free bees were inoculated with mono or dual BGM isolates at age=1 day. Controls included GF=germ free and DH5a=E coli strain DH5a. At age=6 days, bees were fed 5 μl of Serratia marcescens KZ-11 (“SnM” modified for Kanamicin resistance, at OD₆₀₀=0.5.) At age=9 days guts were homogenized in 200 μl PBS and dilutions were plated on HIA+5% SB+Kan 50 μg/ml.) Colonies were counted after over-night incubation. The defined community inoculated bees (DC) and wKB2+Firm5 inoculated bees had significantly reduced populations of KZ11 “SnM” (FIG. 14). The B2 and Firm-5 alone inoculations had no appreciable effect.

### Example 2

### Pathogen Challenge

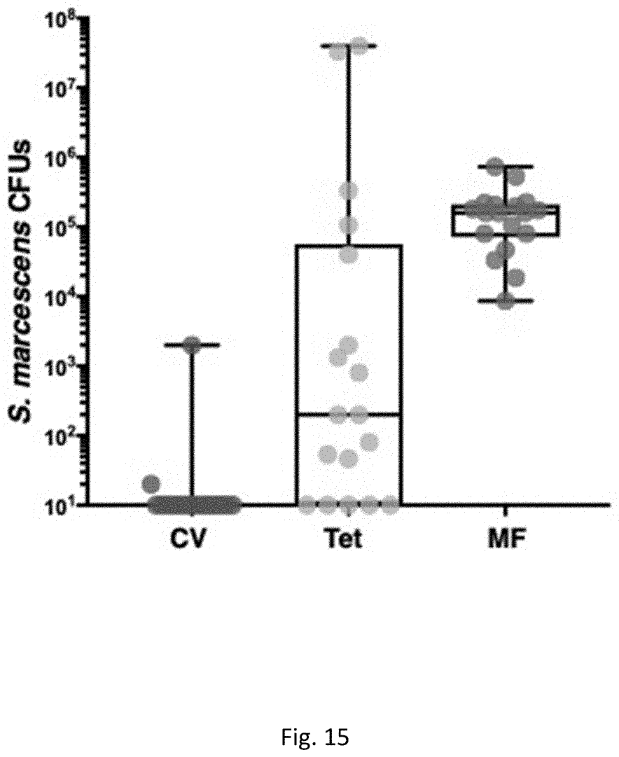

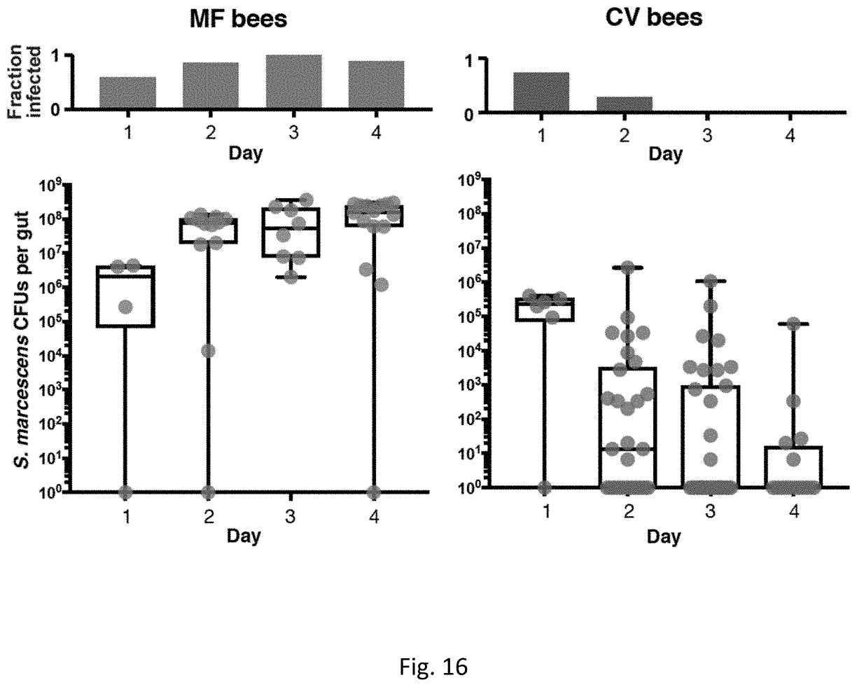

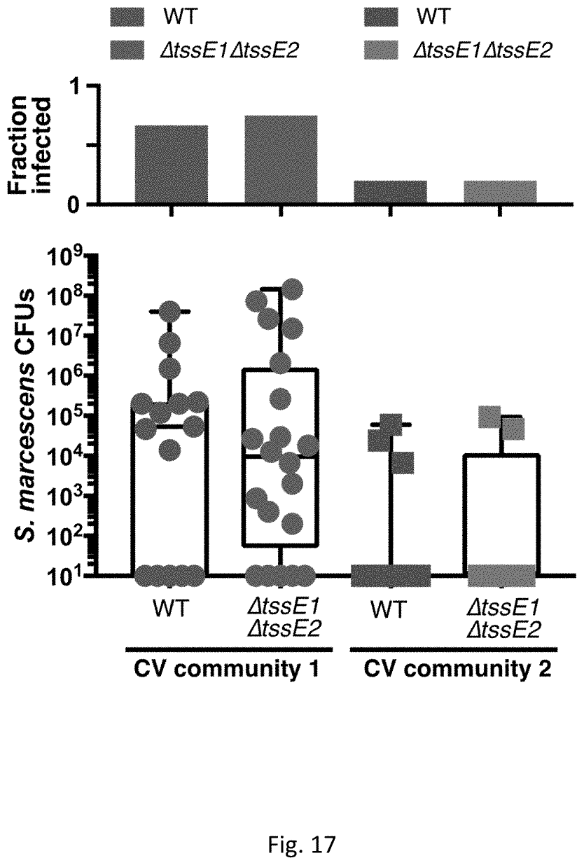

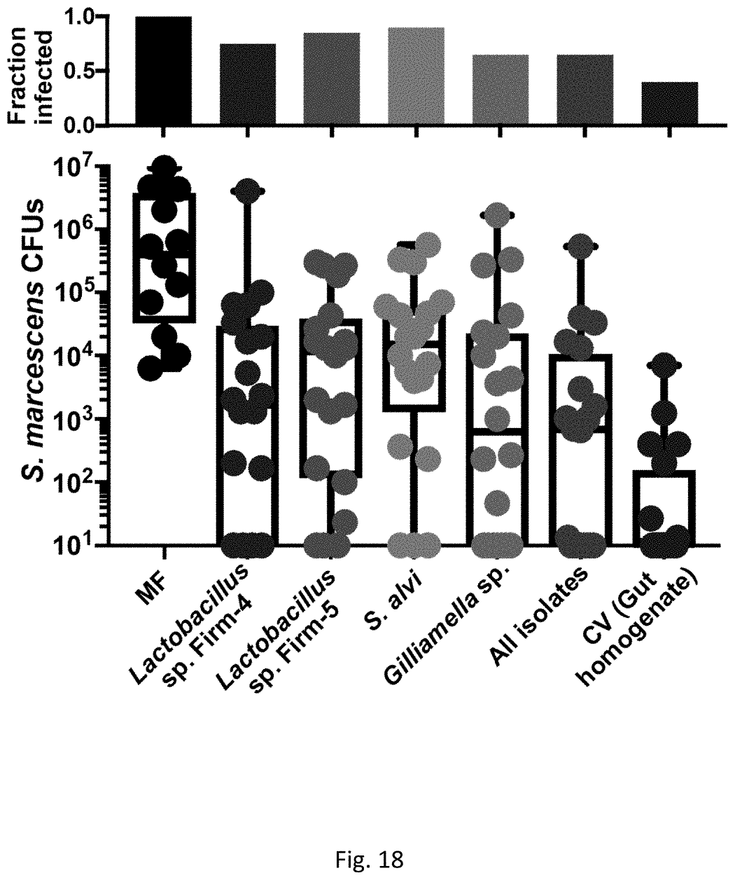

Microbiota in the bee gut provides protection against infectious bacteria, but antibiotics disrupt this protection (FIG. 15-16). Different conventional gut communities (from different hives) give different levels of protection (FIG. 17). This implies that the strains make a difference. A combo of 4 Gilliamella strains shows protective effects against Serratia, and all isolates together gives substantial protection (FIG. 18).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A bacterial co-culture comprising at least two bacterial strains selected from the group consisting of Snodgrassella alvi, Gilliamella apicola, Gilliamella apis, Bartonella apis, Lactobacillus spp., and Bifidobacterium spp.

2. The bacterial co-culture of claim 1, comprising at least two bacterial strains selected from the group consisting of Snodgrassella alvi wkB2, Gilliamella apicola wkB1, Gilliamella apicola wkB 7, Gilliamella apicola PEB0154, Gilliamella apis PEB0162, Gilliamella apis PEB0183, Bartonella apis PEB0150, Lactobacillus “Firm-5” wkB10, Lactobacillus “Firm-5” wkB8 and Bifidobacterium asteroides LCep5.

3. A composition comprising an effective amount of at least two bacterial strains selected from the group consisting of Snodgrassella alvi, Gilliamella apicola, Gilliamella apis, Bartonella apis, Lactobacillus spp., and Bifidobacterium spp., and a carrier.

4. The composition of claim 3 including at least 10³ viable bacteria cells per gram.

5. The composition of claim 3 including at least 10⁶ viable bacteria cells per gram.

6. The composition of claim 3, wherein at least one bacterial strain is in a sporulated form.

7. The composition of claim 3, wherein at least one bacterial strain is provided in a lyophilized form.

8. The composition of claim 3 further comprising an antibiotic.

9. An ingestible composition or supplement for bees comprising an effective amount of two bacterial strains selected from the group consisting of Snodgrassella alvi, Gilliamella apicola, Gilliamella apis, Bartonella apis, Lactobacillus spp., and Bifidobacterium spp. and a carrier suitable for bee consumption.

10. The ingestible composition of claim 9, comprising at least two bacterial strains selected from the group consisting of Snodgrassella alvi wkB2, Gilliamella apicola wkB1, Gilliamella apicola wkB7, Gilliamella apicola PEB0154, Gilliamella apis PEB0162, Gilliamella apis PEB0183, Bartonella apis PEB0150, Lactobacillus “Firm-5” wkB10, Lactobacillus “Firm-5” wkB8 and Bifidobacterium asteroides LCep5.

11. The ingestible composition of claim 9, wherein said ingestible composition is selected from the group consisting of a pollen feed, a sucrose solution and a corn syrup solution.

12. A method of treating or preventing a disease or disorder in a bee or bee colony, the method comprising administering to a bee or bee colony in need thereof a therapeutically effective amount of bacterial co-culture comprising at least two bacterial strains selected from the group consisting of Snodgrassella alvi, Gilliamella apicola, Gilliamella apis, Bartonella apis, Lactobacillus spp., and Bifidobacterium spp.

13. The method of claim 12, wherein said administering is effected at a concentration of said bacterial co-culture comprising between 10³ and 10¹⁰ viable cells in one dose.

14. The method of claim 12, wherein the disorder is colony collapse disorder.

15. The method of claim 12, wherein the disorder is associated with a disruption of the normal gut microbiota due to exposure to a stress such as a chemical, temperature or nutritional stress or a viral, bacterial, fungal or protozoan.

16. A method of promoting health of a bee or bee colony, the method comprising administering to the bee or bee colony a bacterial co-culture of claim 1.

17. An article-of-manufacture comprising packaging material and a composition for treating or preventing a disease or disorder in a bee or bee colony being contained within said packaging material, said composition comprising a bacterial co-culture of claim 1.

18. A method of restoring a bee gut microbiome following a disruptive event, the method comprising administering to the bee or bee colony a bacterial co-culture of claim 1.

19. The method of claim 18, wherein the disruptive event is administration of an antibiotic to the bee or bee colony.
